SEQUENCE LISTING (29 sequences)

SEQ ID NO: 1 - protein, 356 aa, homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 2 - DNA, 20 bp, artificial
  synthetic oligonucleotide primer
ggagcgagaccccactaaca

SEQ ID NO: 3 - DNA, 20 bp, artificial
  synthetic oligonucleotide primer
acatactcagcaccggcctc

SEQ ID NO: 4 - DNA, 21 bp, artificial
  synthetic oligonucleotide primer
aaagccccctaactgactgca

SEQ ID NO: 5 - DNA, 19 bp, artificial
  synthetic oligonucleotide primer
tcaaactcggcggatggtt

SEQ ID NO: 6 - DNA, 20 bp, artificial
  synthetic oligonucleotide primer
ttcagccacacctttccagc

SEQ ID NO: 7 - DNA, 20 bp, artificial
  synthetic oligonucleotide primer
ccttagaggaggcctgggag

SEQ ID NO: 8 - DNA, 21 bp, artificial
  synthetic oligonucleotide primer
aacttgatccctgccccatct

SEQ ID NO: 9 - DNA, 19 bp, artificial
  synthetic oligonucleotide primer
tttctggaccgcattgcct

SEQ ID NO: 10 - DNA, 20 bp, artificial
  synthetic oligonucleotide primer
ggggtcactgtctgaccact

SEQ ID NO: 11 - DNA, 20 bp, artificial
  synthetic oligonucleotide primer
gggaaacctgggatgagatt

SEQ ID NO: 12 - DNA, 20 bp, artificial
  synthetic oligonucleotide primer
acagccccctctgaattctt

SEQ ID NO: 13 - DNA, 20 bp, artificial
  synthetic oligonucleotide primer
ggatgctctccaagttgctc

SEQ ID NO: 14 - DNA, 21 bp, artificial
  synthetic oligonucleotide primer
caggacattgccttcgcctat

SEQ ID NO: 15 - DNA, 21 bp, artificial
  synthetic oligonucleotide primer
taggcccaaaatcaccgtctc

SEQ ID NO: 16 - DNA, 20 bp, artificial
  synthetic oligonucleotide primer
cgtgagcgatgagaaggaca

SEQ ID NO: 17 - DNA, 20 bp, artificial
  synthetic oligonucleotide primer
cgatctgtgcttggttgtgc

SEQ ID NO: 18 - DNA, 21 bp, artificial
  synthetic oligonucleotide primer
cggccagacactttcatcaga

SEQ ID NO: 19 - DNA, 22 bp, artificial
  synthetic oligonucleotide primer
tggattcatcgcttgtgtcttg

SEQ ID NO: 20 - DNA, 20 bp, artificial
  synthetic oligonucleotide primer
cctctggctgtggacaaaat

SEQ ID NO: 21 - DNA, 20 bp, artificial
  synthetic oligonucleotide primer
ctgctcacaagaagcagtgg

SEQ ID NO: 22 - DNA, 20 bp, artificial
  synthetic oligonucleotide primer
gcccaagcatattgtggagt

SEQ ID NO: 23 - DNA, 20 bp, artificial
  synthetic oligonucleotide primer
agggtaggatgtggcacaag

SEQ ID NO: 24 - DNA, 20 bp, artificial
  synthetic oligonucleotide primer
ttgaagttgacggaccccaa

SEQ ID NO: 25 - DNA, 19 bp, artificial
  synthetic oligonucleotide primer
tgttgatgtgctgctgcga

SEQ ID NO: 26 - DNA, 21 bp, artificial
  synthetic oligonucleotide primer
ttccatccagttgccttcttg

SEQ ID NO: 27 - DNA, 21 bp, artificial
  synthetic oligonucleotide primer
catttccacgatttcccagag

SEQ ID NO: 28 - DNA, 21 bp, artificial
  synthetic oligonucleotide primer
cacaaaccaccaagtggagga

SEQ ID NO: 29 - DNA, 21 bp, artificial
  synthetic oligonucleotide primer
acaaggtacaacccatcggct

(19) United States
(12) Patent Application Publication
(10) Pub. No.: US 2021/0032300 A1
(43) Pub. Date: Feb. 4, 2021

(54) METHODS AND MATERIALS FOR TREATING BRAIN INJURIES

(71) Applicant: The Penn State Research Foundation, University Park, PA (US)

(72) Inventors: Gong Chen, State College, PA (US); Lei Zhang, University Park, PA (US); Ziyuan Guo, State College, PA (US); Zifei Pei, University Park, PA (US)

(21) Appl. No.: 16/966,691

(22) PCT Filed: Feb. 1, 2019
(86) PCT No.: PCT/US2019/016378

§ 371 Date: Jul. 31, 2020

Related U.S. Application Data
(60) Provisional application No. 62/625,533, filed on Feb. 2, 2018.

(51) Int. Cl. C07K 14/47; A61P 25/00; C12N 15/86; C12N 7/00; A61K 9/00; C12N 9/12

(52) U.S. Cl. CPC C07K 14/4705; A61P 25/00; C12N 15/86; A61K 48/00; A61K 9/0085; C12N 9/1241; C12Y 207/07; C12N 7/00

(57) ABSTRACT

This document provides methods and materials for treating brain injuries. For example, methods and materials for using nucleic acid encoding a NeuroD1 polypeptide to convert reactive astrocytes within a brain (e.g., cerebral cortex) into functional neurons (e.g., neurons that can be functionally integrated into the brain of a living mammal (e.g., a human)) are provided.

### CROSS-REFERENCE TO RELATED APPLICATIONS

This application claims the benefit of U.S. Patent Application Ser. No. 62/625,533, filed on Feb. 2, 2018. The disclosure of the prior application is considered part of (and is incorporated by reference in) the disclosure of this application.

### STATEMENT REGARDING FEDERAL FUNDING

This invention was made with government support under AG045656 and MH083911 awarded by the National Institutes of Health. The government has certain rights in the invention.

### BACKGROUND

### 1. Technical Field

This document relates to methods and materials for treating brain injuries in a mammal. For example, this document relates to methods and materials for using nucleic acid encoding a neuronal differentiation 1 (NeuroD1) polypeptide to convert reactive astrocytes in a brain (e.g., cerebral cortex of the brain) into functional neurons (e.g., to rebalance neuron:glia ratio) within the brain of a living mammal (e.g., a human).

### 2. Background Information

The central nervous system (CNS) includes both neurons and glial cells, forming a delicate balance to maintain normal brain functions. CNS injury is often studied in the context of either neuronal loss or glial scar. Generating new neurons after nerve injury in the adult mammalian CNS is difficult despite decades of research (Cregg et al., 2014 Exp Neurol 253:197-207; He et al., 2016 Neuron 90:437-451; and Yiu et al., 2006 Nat Rev Neurosci 7:617-627). Glial scar not only serves as a physical barrier but also a chemical barrier for neuroregeneration by accumulating neuroinhibitory factors such as chondroitin sulfate proteoglycans (CSPGs) and lipocalin-2 (LCN2), as well as inflammatory cytokines such as TNFα and interleukin-1β (IL-1β) (Ferreira et al., 2015 Prog Neurobiol 131:120-136; Koprivica et al., 2005 Science 310:106-110; and Silver et al., 2004 Nat Rev Neurosci 5:146-156).

### SUMMARY

This document provides methods and materials for generating functional neurons within a brain. For example, this document provides methods and materials for using nucleic acid encoding a NeuroD1 polypeptide to convert astrocytes (e.g., reactive astrocytes) within a brain (e.g., cerebral cortex) into functional neurons (e.g., neurons that can be functionally integrated into the brain of the living mammal (e.g., a human)). In some cases, the materials and methods provided herein can be used to treat brain injury. For example, NeuroD1-mediated astrocyte-to-neuron (AtN) conversion can be used to treat a brain injury (e.g., following a brain injury) by converting reactive astrocytes into functional neurons, rebalancing neuron:glia ratios, repairing damaged brain tissue (e.g., repairing glial scar tissue by, for example, reversing glial scar tissue back to neural tissue), reducing neuroinflammation, restoring the blood-brain-barrier, transforming A1 astrocytes (e.g., transforming toxic A1 astrocytes into less harmful astrocytes), and/or reducing the amount of toxic M1 microglia

As demonstrated herein, glia-to-neuron conversion rebalances neuron-glia ratios and reverses glial scar back to neural tissue. Ectopic expression of NeuroD1 in reactive astrocytes in the motor cortex reduced glial reactivity and transformed toxic A1 astrocytes into less harmful astrocytes before neuronal conversion. Converting reactive astrocytes into neurons reduced microglia-mediated neuroinflammation, restored the blood-brain-barrier, and restored synaptic density in injury sites.

Nerve injury often causes neuronal loss and glial proliferation, disrupting the delicate balance between neurons and glial cells in the brain. Thus, having the ability to convert reactive astrocytes into functional neurons as described herein can provide a unique and unrealized opportunity to treat brain injuries.

In general, one aspect of this document features a method for repairing glial scar tissue in a cerebral cortex of a living mammal's brain. The method can include, or consist essentially of, administering nucleic acid encoding a NeuroD1 polypeptide to astrocytes within the cerebral cortex of a living mammal's brain, where the NeuroD1 polypeptide is expressed by the astrocytes, where the astrocytes form functional neurons within the cerebral cortex, and where the glial scar tissue in the living mammal's brain is reversed back to neural tissue. The cerebral cortex, after administration of the nucleic acid encoding a NeuroD1 polypeptide, can have decreased expression of glial fibrillary acidic protein (Gfap), lipocalin-2 (Lcn2), and/or chondroitin sulfate proteoglycan (CSPG). The cerebral cortex, after administration of the nucleic acid encoding a NeuroD1 polypeptide, can have increased expression of annexin A2 (Anax2), thrombospondin 1 (Thbs1), glypican 6 (Gpc6), and/or brain-derived neurotrophic factor (Bdnf). The mammal can be a human. The astrocytes can be reactive astrocytes. The NeuroD1 polypeptide can be a human NeuroD1 polypeptide. The cerebral cortex can be a motor cortex. The nucleic acid encoding the NeuroD1 polypeptide can be administered to the astrocytes in the form of a viral vector. The viral vector can be an adeno-associated viral vector (e.g., an adeno-associated virus serotype 9 viral vector). The method also can include administering a nucleic acid encoding a recombinase to astrocytes within the cerebral cortex, where the recombinase polypeptide is expressed by the astrocytes, and where the nucleic acid encoding a NeuroD1 polypeptide is flanked by recombinase target sites. The recombinase can be a Cre recombinase, and the recombinase target sites can be LoxP sites. The nucleic acid encoding a NeuroD1 polypeptide can be an inverted nucleic acid sequence. The nucleic acid encoding the recombinase can be operably linked to a promoter sequence, such as an astrocyte-specific promoter sequence. The astrocyte-specific promoter sequence can include a GFAP promoter sequence. The nucleic acid encoding the NeuroD1 polypeptide can be operably linked to a promoter sequence, such as a constitutive promoter sequence. The constitutive promoter sequence can include a CAG promoter sequence. The administration can include a direct injection into the cerebral cortex of the living mammal's brain. The administration can include an intracranial, intrathecal, intraperitoneal, intravenous, intranasal, intramuscular, or oral administration.

In another aspect, this document features a method for rebalancing the neuron:glia ratio in a cerebral cortex of a living mammal's brain. The method can include, or consist essentially of, administering nucleic acid encoding a NeuroD1 polypeptide to astrocytes within the cerebral cortex of a living mammal's brain, where the NeuroD1 polypeptide is expressed by the astrocytes, where the astrocytes form functional neurons within the cerebral cortex, and where the neuron:glia ratio in the living mammal's brain is increased. The neuron:glia ratio can be increased by decreasing the number of astrocytes. The neuron:glia ratio can be increased by increasing the number of neurons. The neuron:glia ratio can be increased by both decreasing the number of astrocytes and by increasing the number of neurons. The mammal can be a human. The astrocytes can be reactive astrocytes. The NeuroD1 polypeptide can be a human NeuroD1 polypeptide. The cerebral cortex can be a motor cortex. The nucleic acid encoding the NeuroD1 polypeptide can be administered to the astrocytes in the form of a viral vector. The viral vector can be an adeno-associated viral vector (e.g., an adeno-associated virus serotype 9 viral vector). The method also can include administering a nucleic acid encoding a recombinase to astrocytes within the cerebral cortex, where the recombinase polypeptide is expressed by the astrocytes, and where the nucleic acid encoding a NeuroD1 polypeptide is flanked by recombinase target sites. The recombinase can be a Cre recombinase, and the recombinase target sites can be LoxP sites. The nucleic acid encoding a NeuroD1 polypeptide can be an inverted nucleic acid sequence. The nucleic acid encoding the recombinase can be operably linked to a promoter sequence, such as an astrocyte-specific promoter sequence. The astrocyte-specific promoter sequence can include a GFAP promoter sequence. The nucleic acid encoding the NeuroD1 polypeptide can be operably linked to a promoter sequence, such as a constitutive promoter sequence. The constitutive promoter sequence can include a CAG promoter sequence. The administration can include a direct injection into the cerebral cortex of the living mammal's brain. The administration can include an intracranial, intrathecal, intraperitoneal, intravenous, intranasal, intramuscular, or oral administration.

In another aspect, this document features a method for reducing neuroinflammation in a cerebral cortex of a living mammal's brain. The method can include, or consist essentially of, administering nucleic acid encoding a NeuroD1 polypeptide to astrocytes within the cerebral cortex of a living mammal's brain, where the NeuroD1 polypeptide is expressed by the astrocytes, where the astrocytes form functional neurons within the cerebral cortex, and where neuroinflammation in the living mammal's brain is reduced. The cerebral cortex, after administration of the nucleic acid encoding a NeuroD1 polypeptide, can have decreased expression of tumor necrosis factor alpha (TNFa), interleukin 1 beta (IL-1b), and/or cluster of designation 68 (CD68). The mammal can be a human. The astrocytes can be reactive astrocytes. The NeuroD1 polypeptide can be a human NeuroD1 polypeptide. The cerebral cortex can be a motor cortex. The nucleic acid encoding the NeuroD1 polypeptide can be administered to the astrocytes in the form of a viral vector. The viral vector can be an adeno-associated viral vector (e.g., an adeno-associated virus serotype 9 viral vector). The method also can include administering a nucleic acid encoding a recombinase to astrocytes within the cerebral cortex, where the recombinase polypeptide is expressed by the astrocytes, and where the nucleic acid encoding a NeuroD1 polypeptide is flanked by recombinase target sites. The recombinase can be a Cre recombinase, and the recombinase target sites can be LoxP sites. The nucleic acid encoding a NeuroD1 polypeptide can be an inverted nucleic acid sequence. The nucleic acid encoding the recombinase can be operably linked to a promoter sequence, such as an astrocyte-specific promoter sequence. The astrocyte-specific promoter sequence can include a GFAP promoter sequence. The nucleic acid encoding the NeuroD1 polypeptide can be operably linked to a promoter sequence, such as a constitutive promoter sequence. The constitutive promoter sequence can include a CAG promoter sequence. The administration can include a direct injection into the cerebral cortex of the living mammal's brain. The administration can include an intracranial, intrathecal, intraperitoneal, intravenous, intranasal, intramuscular, or oral administration.

In another aspect, this document features a method for restoring the blood-brain-barrier in a cerebral cortex of a living mammal's brain. The method can include, or consist essentially of, administering nucleic acid encoding a NeuroD1 polypeptide to astrocytes within the cerebral cortex of a living mammal's brain, where the NeuroD1 polypeptide is expressed by the astrocytes, where the astrocytes form functional neurons within the cerebral cortex, and where the blood-brain-barrier in the living mammal's brain is restored. The cerebral cortex, after administration of the nucleic acid encoding a NeuroD1 polypeptide, can have increased AQP4 signaling with blood vessels. The mammal can be a human. The astrocytes can be reactive astrocytes. The NeuroD1 polypeptide can be a human NeuroD1 polypeptide. The cerebral cortex can be a motor cortex. The nucleic acid encoding the NeuroD1 polypeptide can be administered to the astrocytes in the form of a viral vector. The viral vector can be an adeno-associated viral vector (e.g., an adeno-associated virus serotype 9 viral vector). The method also can include administering a nucleic acid encoding a recombinase to astrocytes within the cerebral cortex, where the recombinase polypeptide is expressed by the astrocytes, and where the nucleic acid encoding a NeuroD1 polypeptide is flanked by recombinase target sites. The recombinase can be a Cre recombinase, and the recombinase target sites can be LoxP sites. The nucleic acid encoding a NeuroD1 polypeptide can be an inverted nucleic acid sequence. The nucleic acid encoding the recombinase can be operably linked to a promoter sequence, such as an astrocyte-specific promoter sequence. The astrocyte-specific promoter sequence can include a GFAP promoter sequence. The nucleic acid encoding the NeuroD1 polypeptide can be operably linked to a promoter sequence, such as a constitutive promoter sequence. The constitutive promoter sequence can include a CAG promoter sequence. The administration can include a direct injection into the cerebral cortex of the living mammal's brain. The administration can include an intracranial, intrathecal, intraperitoneal, intravenous, intranasal, intramuscular, or oral administration.

In another aspect, this document features a method for transforming an A1 astrocyte in a cerebral cortex of a living mammal's brain. The method can include, or consist essentially of, administering nucleic acid encoding a NeuroD1 polypeptide to astrocytes within the cerebral cortex, where the NeuroD1 polypeptide is expressed by the astrocytes, where the astrocytes form functional neurons within the cerebral cortex, and where the A1 astrocyte is transformed into a less harmful astrocyte. The cerebral cortex, after administration of the nucleic acid encoding a NeuroD1 polypeptide, can have decreased expression of guanylate Binding Protein 2 (Gbp2) and/or serpin family G member 1 (Serping1). The mammal can be a human. The astrocytes can be reactive astrocytes. The NeuroD1 polypeptide can be a human NeuroD1 polypeptide. The cerebral cortex can be a motor cortex. The nucleic acid encoding the NeuroD1 polypeptide can be administered to the astrocytes in the form of a viral vector. The viral vector can be an adeno-associated viral vector (e.g., an adeno-associated virus serotype 9 viral vector). The method also can include administering a nucleic acid encoding a recombinase to astrocytes within the cerebral cortex, where the recombinase polypeptide is expressed by the astrocytes, and where the nucleic acid encoding a NeuroD1 polypeptide is flanked by recombinase target sites. The recombinase can be a Cre recombinase, and the recombinase target sites can be LoxP sites. The nucleic acid encoding a NeuroD1 polypeptide can be an inverted nucleic acid sequence. The nucleic acid encoding the recombinase can be operably linked to a promoter sequence, such as an astrocyte-specific promoter sequence. The astrocyte-specific promoter sequence can include a GFAP promoter sequence. The nucleic acid encoding the NeuroD1 polypeptide can be operably linked to a promoter sequence, such as a constitutive promoter sequence. The constitutive promoter sequence can include a CAG promoter sequence. The administration can include a direct injection into the cerebral cortex of the living mammal's brain. The administration can include an intracranial, intrathecal, intraperitoneal, intravenous, intranasal, intramuscular, or oral administration.

In another aspect, this document features a method for reducing the amount of toxic M1 microglia in a cerebral cortex of a living mammal's brain. The method can include, or consist essentially of, administering nucleic acid encoding a NeuroD1 polypeptide to astrocytes within the cerebral cortex of a living mammal's brain, where the NeuroD1 polypeptide is expressed by the astrocytes, where the astrocytes form functional neurons within the cerebral cortex, and where the amount of toxic M1 microglia in the living mammal's brain is reduced. The toxic M1 microglia, after administration of the nucleic acid encoding a NeuroD1 polypeptide, can have the morphology of resting microglia. The mammal can be a human. The astrocytes can be reactive astrocytes. The NeuroD1 polypeptide can be a human NeuroD1 polypeptide. The cerebral cortex can be a motor cortex. The nucleic acid encoding the NeuroD1 polypeptide can be administered to the astrocytes in the form of a viral vector. The viral vector can be an adeno-associated viral vector (e.g., an adeno-associated virus serotype 9 viral vector). The method also can include administering a nucleic acid encoding a recombinase to astrocytes within the cerebral cortex, where the recombinase polypeptide is expressed by the astrocytes, and where the nucleic acid encoding a NeuroD1 polypeptide is flanked by recombinase target sites. The recombinase can be a Cre recombinase, and the recombinase target sites can be LoxP sites. The nucleic acid encoding a NeuroD1 polypeptide can be an inverted nucleic acid sequence. The nucleic acid encoding the recombinase can be operably linked to a promoter sequence, such as an astrocyte-specific promoter sequence. The astrocyte-specific promoter sequence can include a GFAP promoter sequence. The nucleic acid encoding the NeuroD1 polypeptide can be operably linked to a promoter sequence, such as a constitutive promoter sequence. The constitutive promoter sequence can include a CAG promoter sequence. The administration can include a direct injection into the cerebral cortex of the living mammal's brain. The administration can include an intracranial, intrathecal, intraperitoneal, intravenous, intranasal, intramuscular, or oral administration.

In another aspect, this document features a composition for forming functional neurons in a cerebral cortex of a living mammal's brain. The composition can include, or consist essentially of, a nucleic acid vector including an inverted nucleic acid sequence encoding a NeuroD1 polypeptide flanked by recombinase target sites, and a nucleic acid vector including a nucleic acid sequence encoding a recombinase. The nucleic acid vector including an inverted nucleic acid sequence encoding a NeuroD1 polypeptide flanked by recombinase target sites can be a viral vector. The viral vector can be an adeno-associated viral vector (e.g., an adeno-associated virus serotype 9 viral vector). The inverted nucleic acid sequence encoding the NeuroD1 polypeptide can be operably linked to a promoter sequence, such as a constitutive promoter sequence. The constitutive promoter sequence can include a CAG promoter sequence. The nucleic acid vector including a nucleic acid sequence encoding a recombinase can be a viral vector. The viral vector is an adeno-associated viral vector (e.g., an adeno-associated virus serotype 9 viral vector). The nucleic acid sequence encoding the recombinase can be operably linked to a promoter sequence, such as an astrocyte-specific promoter sequence. The astrocyte-specific promoter sequence can include a GFAP promoter sequence. The recombinase can be a Cre recombinase, and the recombinase target sites can be LoxP sites.

In another aspect, this document features a vector for forming functional neurons in a cerebral cortex of a living mammal's brain. The vector can include, or consist essentially of, an inverted nucleic acid sequence encoding a NeuroD1 polypeptide flanked by recombinase target sites, and a nucleic acid sequence encoding a recombinase. The vector can be a viral vector. The viral vector can be an adeno-associated viral vector (e.g., an adeno-associated virus serotype 9 viral vector). The inverted nucleic acid sequence encoding the NeuroD1 polypeptide can be operably linked to a promoter sequence, such as a constitutive promoter sequence. The constitutive promoter sequence can include a CAG promoter sequence. The nucleic acid sequence encoding the recombinase can be operably linked to a promoter sequence, such as an astrocyte-specific promoter sequence. The astrocyte-specific promoter sequence can include a GFAP promoter sequence. The recombinase can be a Cre recombinase, and the recombinase target sites can be LoxP sites.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Significant proliferation of astrocytes after severe stab injury. (a) Severe stab injury in mouse motor cortex induced reactive astrocytes and tissue loss. Upper row illustrates normal astrocytes in non-injured cortical tissue of Glial fibrillary acidic protein (GFAP)::GFP mice with elaborate processes and non-overlapping with their neighboring astrocytes. Bottom row shows a significant tissue loss induced by stab injury in the mouse motor cortex and a large number of hypertrophic reactive astrocytes at 10 days post stab injury (dps). Scale bars=100 μm (left low magnification (mag) panels), 20 μm (right high mag panels). Right bar graph, quantitative analysis shows an increase of astrocytic number after stab injury. NI, non-injury. n=4 mice. ***P<0.001, Student's t-test. (b) Proliferation of astrocytes after stab injury. 5-Bromo-2′-deoxyuridine (BrdU) was injected intraperitoneally into GFAP::GFP mice daily until 10 dps. Many GFP⁺ cells were co-labeled with BrdU (which stained red) and GFAP (which stained cyan), indicating cell division of astrocytes after stab injury. Quantitative analysis found that 38.7±2.5% of GFP+ astrocytes were BrdU+, suggesting a high proliferation rate in the injury sites. n=4 mice. Scale bars=50 μm (top left), and 10 μm (top right and bottom left).

FIG. 2. Overexpression of NeuroD1 by retroviruses efficiently converted reactive astrocytes into neurons after stab injury. Retroviruses carrying CAG::NeuroD1-IRES-GFP or CAG::GFP (control) were injected into stab-injured motor cortex at 4 dps. At 14 days post viral injection (dpi), mice were sacrificed and subjected to immunostaining. The GFP-infected cells showed glial morphology and immunonegative for NeuN (top row), whereas the majority of NeuroD1-infected cells were NeuN⁺ neurons (bottom row). Right bar graph shows that about 90% of NeuroD1-infected cells were converted into neurons. Scale bar=10 μm.

FIG. 3. Schematic illustration shows the working model of AAV9 Cre-FLEX system. GFAP::Cre viruses express Cre in astrocytes, where Cre acts at the loxP-type recombination sites of FLEX-CAG::NeuroD1-P2A-mCherry (inverted sequence). After Cre-mediated recombination, NeuroD1 is expressed under the control of CAG promoter.

FIG. 4. Rescue of neuron:astrocyte ratio through NeuroD1-mediated in vivo AtN conversion. (a) Schematic illustration shows stab injury and AAV injection in mouse motor cortex, followed by analyses at different time points. To evaluate the effect of NeuroD1-treatment, the adeno-associated virus serotype 9 (AAV9) carrying GFAP::Cre and FLEX-CAG::NeuroD1-P2A-mCherry or FLEX-CAG::mCherry-P2A-mCherry (control) were injected into the injury site at 4 days post stab injury (dps). Mice were sacrificed at 3, 7, or 14 days post viral injection (dpi) for analyses. A brain section with NeuroD1-AAV9 injection showed broad viral infection around the motor cortex area (top right). (b) NeuroD1 efficiently converted reactive astrocytes into neurons in stab-injured brains. Low-magnification images (left panels) show the efficient viral infection in stab-injured cortex. Scale bar=100 μm. High-magnification images (right panels) reveal the control-AAV infected cells with clear astrocytic morphology (7 dpi), while NeuroD1-AAV infected cells (arrowheads) show neuronal morphology with high expression level of NeuroD1 (which stained green). Co-immunostaining of NeuN and GFAP confirmed that the mCherry control-AAV infected cells (which stained red) were GFAP⁺ astrocytes (top row, which stained cyan), whereas most NeuroD1-AAV infected cells (bottom row, arrow heads) were NeuN⁺ neurons (which stained magenta). The astrocytic morphology in the NeuroD1 group also became less hypertrophic compared to the control group. Scale bar=20 μm. (c) Astrocytes not depleted in NeuroD1-converted areas. Control AAV-infected injury areas showed intensive GFAP signals (which stained green) with hypertrophic morphology (top row, 7 dpi). In contrast, NeuroD1-infected injury area showed significantly reduced GFAP expression, and astrocytic morphology was less reactive but closer to healthy ones (bottom row, arrow head). Importantly, astrocytes persisted in the area with many NeuroD1-converted neurons (which stained red). Scale bar=200 μm (low magnification), and 20 μm (high magnification). Quantitative analysis revealed a significant reduction of both GFAP intensity and GFAP-covered area in NeuroD1-infected injury areas (right bar graphs). The GFAP signal in NeuroD1 group was reduced to half of control group but still higher than the non-injured brains, indicating that astrocytes were not depleted after NeuroD1 conversion. n=4-6 mice per group. **P<0.01, ***P<0.001, two-way ANOVA followed by Bonferroni post-hoc test. (d) Increased proliferation of astrocytes after NeuroD1-mediated cell conversion. BrdU was applied daily in GFAP::GFP mice between 7-14 days post viral injection, a time window of cell conversion, to assess cell proliferation. Many proliferating astrocytes that were co-labeled with BrdU (which stained magenta) and GFP (which stained green) were detected in the vicinity of NeuroD1-converted neurons (red). Scale bar=20 μm. Quantitative analysis revealed a significant increase of the number of proliferating astrocytes (BrdU⁺/GFAP⁺) in NeuroD1-infected injury areas, compared to the control group. n=3 mice. *P<0.05, Student's t-test. (e) Rescue of neuron:astrocyte ratio after NeuroD1-mediated AtN conversion. Left images illustrate neurons (NeuN, which stained red) and astrocytes (S100β, which stained green; GFAP, which stained cyan) in non-injured brains, mCherry control group, and NeuroD1 group. Scale bar=100 μm. Right bar graphs, quantitative analyses illustrate the number of NeuN⁺ neurons, GFAP⁺ or S100b⁺ astrocytes, and the neuron:astrocyte ratio among non-injured, mCherry control, and NeuroD1 groups. The neuron:astrocyte ratio was measured as 4:1 in non-injured mouse motor cortex, but significantly decreased to 0.6 after stab injury, and then reversed back to 2.6 by NeuroD1-mediated AtN conversion. n=3-6 mice per group. *P<0.05, ***P<0.001, two-way ANOVA plus Sidak's test.

FIG. 5. Quantitative analysis of cell conversion efficiency after viral infection. (a) The majority (90%) of NeuroD1-infected cells were NeuN⁺ but GFAP⁻, whereas control mCherry AAV-infected cells were mostly GFAP⁺ (80%). The NeuroD1-mediated AtN conversion was largely completed at 7-14 dpi. n=4-6 mice per group. ***P<0.001, two-way ANOVA followed with Bonferroni tests. (b) Representative images show the tissue loss in mouse motor cortex after stab injury, which was significantly repaired after NeuroD1 infection. Scale bar=100 μm.

FIG. 6. NeuroD1 transformed A1-type harmful reactive astrocytes in early time point before neuronal conversion. (a) Quantitative real-time PCR (qRT-PCR) analysis revealed a dramatic increase of reactive astrocytic genes Gfap and Lcn2 after stab injury, but significantly attenuated in NeuroD1-infected areas. n=4 mice. **P<0.01, ***P<0.001, one-way ANOVA followed with Sidak's test. (b) Toxic A1 type astrocyte-specific genes guanylate Binding Protein 2 (Gbp2) and serpin family G member 1 (Serping1) were upregulated several hundred fold in stab-injured cortices compared to non-injured cortices, but markedly reduced in NeuroD1-infected cortices. n=4 mice. ***P<0.001, one-way ANOVA followed with Sidak's test. (c) More quantitative analyses with qRT-PCR revealed a significant upregulation of astrocytic functional genes in NeuroD1-infected cortices compared to the mCherry control group. *P<0.05, **P<0.01, ***P<0.001, Student's t-test. (d) Representative images show that in control-AAV infected injury sites. Neural injury marker lipocalin-2 (LCN2) was highly expressed (top row, arrowhead; 3 dpi). In contrast, NeuroD1-infected cells showed much-reduced LCN2 signal (bottom row, arrowheads). Scale bar=20 μm. Right bar graph, quantitative analysis revealed a reduced LCN2 expression in NeuroD1-infected cells compared to the control-AAV infected cells. n=3 mice. *P<0.05, Student's t-test. (e) Representative images revealed in control-AAV infected injury sites that chondroitin sulfate proteoglycan (CSPG) was highly expressed in reactive astrocytes (upper panels, arrowheads; 7 dpi); whereas NeuroD1-infected areas showed significantly reduced CSPG signal (lower panels). Scale bar=200 μm (low mag), 10 μm (high mag). Right bar graph shows quantitative analysis of the CSPG signal in control and NeuroD1 groups. n=3 mice. *P<0.05, Student's t-test.

FIG. 7. Highly efficient expression of NeuroD1 in stab-injured areas using the AAV9 Cre-FLEX system. (a) Representative images (left panels) show widespread AAV infection in the stab-injured cortical areas. Right bar graph, quantitative analysis shows that about 90% of NeuroD1-mCherry infected cells expressed high level of NeuroD1. Scale bar=100 μm. ***P<0.001, one-way ANOVA plus Sidak's test. (b) Representative images show early expression of NeuroD1 in infected astrocytes (3 dpi). Quantitatively, among NeuroD1-mCherry infected cells, 92.8±2.8% are GFAP-positive astrocytes (which stained cyan), and 87.4±2.5% are positive for NeuroD1 (which stained green).

FIG. 8. Early effect of NeuroD1 in reducing GFAP expression after infecting astrocytes. (a) Quantitative real-time PCR (qRT-PCR) confirmed a drastic increase of NeuroD1 expression at 3 days post NeuroD1-AAV infection (dpi). n=4 mice. ***P<0.001, one-way ANOVA plus Sidak's test. (b) Quantitative RT-PCR results suggest no significant changes in the expression of A2-astrocytic markers S100a10 or Tm4f1. n=4 mice. No statistical significance. One-way ANOVA plus Sidak's test. (c) Left images illustrate less reactive morphology and less GFAP expression in NeuroD1-infected astrocytes (bottom row, arrowhead) compared to mCherry-infected astrocytes (top row, arrowhead). Right bar graph showing quantitative result.

FIG. 9. NeuroD1-treatment attenuated microglial inflammatory responses. (a) Microglia (Iba1, which stained green) in non-injured brains displayed ramified branches (top row), but showed hypertrophic amoeboid shape in stab-injured areas (middle row, 7 dpi). In NeuroD1-infected injury areas, however, microglia returned to ramified morphology again (bottom row). Such morphological changes of microglia coincided with the morphological changes of astrocytes (GFAP::GFP labeling in left column). Scale bar=20 μm. (b) Representative images show a close look of the microglia morphology (Iba1, which stained green) contacting mCherry-infected astrocytes (left panel, which stained red) or NeuroD1-mCherry infected astrocytes (right panel, which stained red) at 3 dpi. Microglia showed clear morphological difference when contacting the NeuroD1-infected astrocytes as early as 3 dpi, before neuronal conversion. Scale bar=20 μm. Lower bar graph illustrates that the gene expression level of inflammatory factors tumor necrosis factor alpha (Tnfa) and interleukin 1 beta (Il1b) significantly increased in stab-injured cortices compared to non-injured cortical tissue, but such increase was greatly reduced in NeuroD1-infected injury areas (3 dpi). n=4 mice. *P<0.05, ***P<0.001, one-way ANOVA followed with Sidak's test. (c) Representative images illustrate many inflammatory M1 microglia labeled by nitric oxide synthase (iNOS) with amoeboid morphology in the control-AAV infected injury areas (upper panels, 3 dpi). In contrast, microglia in close contact with the NeuroD1-infected astrocytes showed much lower iNOS expression with ramified morphology (lower panels, arrow; 3 dpi). Scale bar=10 μm. Quantitative analysis shows a significant reduction of iNOS signal of Iba1⁺ cells in close contact with NeuroD1-infected cells. n=3-4 mice per group. ***P<0.001, Student's t-test. (d) Representative images illustrating lack of iNOS signal in non-injured brains (upper panels), but high Iba1 and iNOS signal in stab-injured cortical tissue (middle panels, 7 dpi). However, in NeuroD1-infected cortices, both Iba1 and iNOS signals reduced significantly (bottom panels, 7 dpi). Scale bar=50 μm. Right bar graphs, quantitative analyses show the immunofluorescent signal of Iba1 and iNOS in non-injured (white bar), mCherry control (black bar), or NeuroD1 AAV-infected cortices (gray bar) at 3, 7 and 14 dpi. **P<0.01, ***P<0.001. Two-way ANOVA followed by Bonferroni post-hoc tests. n=5-6 mice per group. (e) Representative images show the immunoreactivity of cluster of designation 68 (CD68), a macrophage marker, significantly reduced in NeuroD1-infected cortical tissues. Scale bar=50 μm. Right bar graph shows quantitative analysis results, which revealed a significant reduction of CD68 fluorescent signal in NeuroD1-infected cortices (gray bar) at 3 and 7 dpi. n=5-6 mice per group. **P<0.01, ***P<0.001, two-way ANOVA plus Bonferroni post-hoc test.

FIG. 10. Activation of microglia and astrocytes after stab injury but lack adult neurogenesis in the mouse cortex. (a) Representative images show the drastic accumulation of microglia (Iba1, which stained red) around the injured cortical areas at 4 and 10 days post stab injury. Astrocytes (GFP, which stained green) were also activated around the injury site. Scale bar=200 μm. (b) Proliferation of both microglia and astrocytes in the stab-injured mouse cortex. BrdU was applied daily after stab injury. Representative images show BrdU (which stained red) labeling in many Iba1⁺ (which stained green) cells, suggesting high proliferation of microglia after injury. Scale bar=20 μm. (c) Left image shows resting microglia (which stained cyan) and astrocytes (which stained green) in non-injured GFAP::GFP mouse cortex. Right image illustrates after stab injury (4 dps), both astrocytes (which stained green) and microglia (Iba1, which stained cyan) were BrdU⁺. (d) Very low internal neuroregeneration capability in the adult mouse cortex after stab injury. BrdU was applied every 2 days for 1 month to label the internal newborn neurons after stab injury. BrdU⁺ cells were rarely co-labeled with NeuN (<1%), indicating very low endogenous adult neurogenesis in the mouse cortex. Scale bar=20 μm.

FIG. 11. Repair of blood vessels and blood-brain-barrier after stab injury through NeuroD1-mediated in vivo cell conversion. (a) Representative images show the astrocyte-vascular unit in non-injured mouse cortex. Astrocytes (which stained green, labeled by GFAP::GFP) send their endfeet wrapping around blood vessels (which stained magenta, labeled by LY6C, a vascular endothelial cell marker). Water channel protein aquaporin 4 (AQP4, which stained blue) was highly concentrating at the astrocytic endfeet in resting state, which wrapped around the blood vessels. Scale bar=20 μm. (b) Representative images in lower magnification (top row) and higher magnification (bottom row) show the blood vessel morphology disrupted by stab injury. In NeuroD1-infected areas, the hypertrophic blood vessel morphology was partially reversed, closer to the ones in non-injured brains. Scale bar=100 μm (low mag), 20 μm (high mag). (c) Top row illustrates mislocalization of AQP4 signal (which stained green) and detachment of astrocytic endfeet from blood vessels (which stained magenta, LY6C) after stab injury. The AQP4 signal was spreading throughout the parenchyma tissue without concentrating around the blood vessels. Bottom row illustrates in NeuroD1-infected areas, AQP4 signal (which stained green) was re-associated with blood vessels (which stained magenta, LY6C). Scale bars=100 μm (low magnification), 20 μm (high magnification). (d) Top row illustrates a significant leakage of biotin into the parenchyma tissue after stab injury, suggesting a disruption of blood-brain-barrier (BBB) integrity (7 dpi). Biotin signal was found not only inside the blood vessels but also outside the blood vessels. Bottom row illustrates that in NeuroD1-infected areas, biotin was mostly confined inside blood vessels, suggesting a restoration of BBB integrity. Scale bars=100 μm (low magnification), 20 μm (high magnification).

FIG. 12. Functional rescue by NeuroD1-mediated AtN conversion. (a) Left images show dendritic marker SMI32 (which stained green) drastically reduced after stab injury (middle panel), but significantly rescued in NeuroD1-infected injury regions (bottom panel). The NeuroD1-converted new neurons (which stained red, bottom panel) were co-labeled by SMI32, indicating the newly generated neurons contributing to neural repair. Right bar graph illustrates SMI32 signal partially recovered at 7 and 14 dpi. Scale bar=20 μm. n=3-4 mice per group. ***P<0.001, two-way ANOVA plus Bonferroni post-hoc test. (b) Rescue of synaptic loss after stab injury by NeuroD1-mediated cell conversion. Left images show both glutamatergic synapses (which stained red, Vglut1) and GABAergic synapses (which stained green, GAD67) significantly reduced after stab injury (middle panel, 30 dpi). However, after NeuroD1-treatment, both glutamatergic and GABAergic synapses showed significant recovery. Scale bar=20 μm. Right bar graphs show quantitative analysis results. Both the number of synaptic puncta and the covered area were rescued in NeuroD1-treated injury areas. n=4-5 mice per group. ***P<0.001, two-way ANOVA plus post-hoc test. (c) Electrophysiological analysis demonstrates that the NeuroD1-converted neurons were functional. Left images show patch-clamp recordings performed on NeuroD1-mCherry-infected cells in cortical slices (30 dpi). Right traces show typical recording of large Na⁺ and K⁺ currents, repetitive action potentials, and excitatory (EPSC) and inhibitory (IPSC) postsynaptic currents. n=15 cells from 3 mice. (d) Rescue of tissue loss by NeuroD1-mediated AtN conversion. Top row illustrates cortical tissue damage induced by stab injury with Niss1 staining in a series of brain sections across the injury core (7 dpi). Bottom row illustrates much less tissue loss in the NeuroD1 group. Scale bar=200 μm. Right line graph shows the quantitative analysis result. Across the entire injury areas, cortical tissue loss was significantly rescued throughout the NeuroD1-infected regions. The tissue areas absent of crystal violet signals were quantified. n=5 mice. **P<0.01, two-way ANOVA followed with Bonferroni post-hoc test.

FIG. 13 is a listing of an amino acid sequence of a human NeuroD1 polypeptide (SEQ ID NO:1).

### DETAILED DESCRIPTION

This document provides methods and materials for generating functional neurons within a brain. For example, this document provides methods and materials for using nucleic acid encoding a NeuroD1 polypeptide to trigger glial cells (e.g., reactive astrocytes) within a brain into forming functional neurons within the brain of the living mammal (e.g., a human). Forming functional neurons as described herein can include converting reactive astrocytes within a brain into functional neurons. The term “functional neuron” as used herein refers to a neuron that is functionally integrated into a brain of a living mammal (e.g., a human). For example, a functional neuron can be a glutamatergic neuron and/or a GABAergic neuron. In some cases, materials and methods provided herein can be used to treat brain injury. For example, NeuroD1-mediated AtN conversion can be used to treat brain injury (e.g., following a brain injury) by converting reactive astrocytes into functional neurons, rebalancing neuron:glia ratios, repairing damaged brain tissue (e.g., repairing glial scar tissue by, for example, reversing glial scar tissue back to neural tissue), reducing neuroinflammation, restoring the blood-brain-barrier, transforming A1 astrocytes (e.g., transforming toxic A1 astrocytes into less harmful astrocytes), and/or reducing the amount of toxic M1 microglia.

Any appropriate mammal can be treated as described herein. Examples of mammals that can be treated as described herein can include, without limitation, humans, monkeys, dogs, cats, cows, horses, pigs, rats, and mice. For example, mammals can be treated as described herein to generate functional neurons in the brain of a living mammal. In some cases, a human having a brain injury can be treated as described herein to generate functional neurons in the human's injured brain. A mammal can be identified as having a brain injury using any appropriate diagnostic technique. For example, neurological examinations, neuroimaging, neuropsychological assessments, electrocardiograms (EKGs), cognition tests, language tests, behavioral tests, blood tests, and/or urine tests can be performed to identify a mammal (e.g. a human) as having a brain injury.

Any type of brain injury can be treated as described herein. In some cases, a brain injury treated as described herein can be an acquired brain injury (ABI). In some cases, a brain injury treated as described herein can be a traumatic brain injury (TBI). Examples of brain injuries that can be treated as described herein include, without limitation, concussions, contusions, coup-contrecoup injuries, diffuse axonal injuries, penetrations, blasts, infections, genetic mutations, and comas. In some cases, a brain injury treated as described herein can include the presence of reactive astrocytes. In some cases, a brain injury treated as described herein can include tissue loss. Examples of causes of brain injuries include, without limitation, trauma, stroke, tumor, infection, substance abuse, hypoxia, anoxia, aneurysm, neurological illness, toxins, embolisms, hematomas, brain hemorrhaging, genetic diseases, and comas.

A brain injury treated as described herein can be in any appropriate location within the brain. For example, a brain injury treated as described herein can be in the cerebral cortex (e.g., the motor cortex, sensory cortex, and association cortex), striatum, hippocampus, thalamus, hypothalamus, amygdla, cerebellum, or brain stem. In some cases, a brain injury treated as described herein can be in the motor cortex of a mammal (e.g., a human).

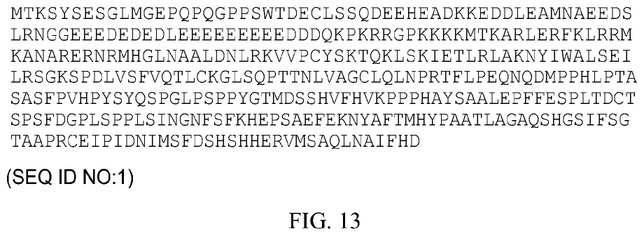

As described herein, a mammal (e.g., a mammal having a brain injury) can be treated by administering nucleic acid designed to express a NeuroD1 polypeptide (e.g., a composition containing nucleic acid designed to express a NeuroD1 polypeptide) to reactive astrocytes within the mammal's brain (e.g., cerebral cortex) in a manner that triggers the reactive astrocytes to form functional neurons. Examples of NeuroD1 polypeptides include, without limitation, those polypeptides having the amino acid sequence set forth in GenBank® accession number NP 002491. In some cases, a NeuroD1 polypeptide can be as set forth in SEQ ID NO:1 (see, e.g., FIG. 13). A NeuroD1 polypeptide can be encoded by a nucleic acid sequence as set forth in GenBank® accession number NM_002500.

Any appropriate method can be used to deliver nucleic acid designed to express a NeuroD1 polypeptide to glial cells (e.g., reactive astrocytes) within the brain of a living mammal. For example, nucleic acid encoding a NeuroD1 polypeptide can be administered to a mammal using one or more vectors such as viral vectors. Vectors for administering nucleic acids (e.g., nucleic acid encoding a NeuroD1 polypeptide) to reactive astrocytes can be prepared using appropriate materials (e.g., packaging cell lines, helper viruses, and vector constructs). See, for example, Gene Therapy Protocols (Methods in Molecular Medicine), edited by Jeffrey R. Morgan, Humana Press, Totowa, N.J. (2002) and Viral Vectors for Gene Therapy: Methods and Protocols, edited by Curtis A. Machida, Humana Press, Totowa, N.J. (2003). In some cases, virus-based vectors can be used to express nucleic acid in dividing cells. In some cases, virus-based vectors can be used to express nucleic acid in non-dividing cells. In some cases, virus-based vectors can be used to express nucleic acid in both dividing cells and non-dividing cells. Virus-based nucleic acid delivery vectors for delivering nucleic acid designed to express a NeuroD1 polypeptide to reactive astrocytes within the brain of a living mammal can be derived from animal viruses, such as adenoviruses, adeno-associated viruses, retroviruses, lentiviruses, vaccinia viruses, herpes viruses, and papilloma viruses. In some cases, nucleic acid encoding a NeuroD1 polypeptide can be delivered to reactive astrocytes using adeno-associated virus vectors (e.g., an adeno-associated virus serotype 2 viral vector, an adeno-associated virus serotype 5 viral vector, or an adeno-associated virus serotype 9 viral vector), lentiviral vectors, retroviral vectors, adenoviral vectors, herpes simplex virus vectors, or poxvirus vector. For example, nucleic acid encoding a NeuroD1 polypeptide can be administered to a mammal using adeno-associated virus vectors to express NeuroD1 polypeptide in both dividing and non-dividing cells.

In addition to nucleic acid encoding a NeuroD1 polypeptide, a viral vector can contain one or more regulatory elements and/or one or more site-specific recombinase elements operably linked to the nucleic acid encoding a NeuroD1 polypeptide. As used herein, “operably linked” refers to positioning of a regulatory element in a vector relative to a nucleic acid in such a way as to permit or facilitate expression of the encoded polypeptide. Examples of regulatory elements can include, without limitation, promoter sequences, enhancer sequences, response elements, signal peptides, internal ribosome entry sequences, polyadenylation signals, terminators, or inducible elements that modulate expression (e.g., transcription or translation) of a nucleic acid. Examples of site-specific recombination elements can include, without limitation, recombinases (e.g., a Cre recombinase), recombination target sites (e.g., LoxP sites), or flip-excision (FLEx) switches that modulate site-specific recombination of a nucleic acid. The choice of element(s) that may be included in a viral vector depends on several factors, including, without limitation, inducibility, targeting, the level of expression desired, and the desired recombination site. For example, a promoter can be included in a viral vector to facilitate transcription of a nucleic acid encoding a NeuroD1 polypeptide. A promoter can be constitutive or inducible (e.g., in the presence of tetracycline), and can affect the expression of a nucleic acid encoding a polypeptide in a general or tissue-specific manner. Examples of cell-specific and/or tissue-specific promoters that can be used to drive expression of a NeuroD1 polypeptide in glial cells (e.g., reactive astrocytes) include, without limitation, NG2, GFAP, Olig2, CAG, EF1a, Aldh1L1, and CMV promoters. In some cases, a CAG promoter can be included in a viral vector to facilitate transcription of a nucleic acid encoding a NeuroD1 polypeptide. For example, loxP-type recombination sites can be included in a viral vector flanking a sequence (e.g., an inverted sequence) of nucleic acid encoding the NeuroD1 polypeptide. In some cases, a GFAP promoter can be included in a viral vector to facilitate transcription of a nucleic acid encoding a recombinase in astrocytes.

In some cases, the methods described herein can be carried out using both nucleic acid encoding a NeuroD1 polypeptide and nucleic acid encoding a recombinase (e.g., a Cre recombinase). In some cases, nucleic acid encoding a NeuroD1 polypeptide and nucleic acid encoding a recombinase can be located on the same viral vector. In such cases, the viral vector can be an adeno-associated viral vector (e.g., an adeno-associated virus serotype 9 viral vector). In some cases, nucleic acid encoding a NeuroD1 polypeptide and nucleic acid encoding a recombinase can be located on separate viral vectors, and each of the separate viral vectors can be administered to glial cells (e.g., reactive astrocytes). In such cases, each of the separate viral vectors can be an adeno-associated viral vector (e.g., an adeno-associated virus serotype 9 viral vector). For example, a first viral vector can contain an inverted sequence of a constitutive CAG promoter operably linked to nucleic acid encoding a NeuroD1 polypeptide where the inverted sequence is flanked by loxP sites, and a second viral vector can contain an astrocyte-specific GFAP promoter operably linked to a Cre recombinase. In this case, the GFAP promoter can drive transcription of Cre recombinase in astrocytes where Cre-mediated recombination leads to high expression of NeuroD1 driven by a strong, constitutive CAG promoter.

In some cases, nucleic acid encoding a NeuroD1 polypeptide (e.g., a composition containing nucleic acid designed to express a NeuroD1 polypeptide) can be administered to a mammal using non-viral vectors. Methods of using non-viral vectors for nucleic acid delivery are described elsewhere. See, for example, Gene Therapy Protocols (Methods in Molecular Medicine), edited by Jeffrey R. Morgan, Humana Press, Totowa, N.J. (2002). For example, nucleic acid encoding a NeuroD1 polypeptide can be administered to a mammal by direct injection of nucleic acid molecules (e.g., plasmids) containing nucleic acid encoding a NeuroD1 polypeptide and, or by administering nucleic acid molecules complexed with lipids, polymers, or nanospheres. In some cases, a genome editing technique such as CRISPR/Cas9-mediated gene editing (see, e.g., U.S. Pat. Nos. 8,697,359; 8,771,945; 8,871,445; 8,795,965; 9,738,908; 9,809,814; 9,803,194; 9,725,714; 9,410,198; and 9,260,752) or TALEN gene editing (see, e.g., U.S. Pat. Nos. 8,586,363; 8,450,471; 8,440,431; and 8,440,432) can be used to introduce exogenous nucleic acid encoding a NeuroD1 polypeptide into cells and/or to activate endogenous NeuroD1 gene expression within cells.

In some cases, delivery of nucleic acid designed to express a NeuroD1 polypeptide to glial cells (e.g., reactive astrocytes) within a brain of a living mammal can result in efficient NeuroD1 expression within the glial cells. For example, from about 10% to about 95% (e.g., from about 10% to about 90%, from about 10% to about 85%, from about 10% to about 80%, from about 10% to about 75%, from about 10% to about 60%, from about 10% to about 50%, from about 10% to about 35%, from about 20% to about 95%, from about 30% to about 95%, from about 40% to about 95%, from about 50% to about 95%, from about 60% to about 95%, from about 70% to about 95%, from about 80% to about 95%, from about 90% to about 95%, from about 20% to about 85%, from about 25% to about 75%, from about 30% to about 65%, or from about 40% to about 55%) of reactive astrocytes infected with a viral vector including nucleic acid sequence encoding a NeuroD1 polypeptide can express NeuroD1. For example, from about 90 to 95 percent (e.g., 92.8±2.8%) of reactive astrocytes infected with a viral vector including nucleic acid sequence encoding a NeuroD1 polypeptide can express NeuroD1.

Nucleic acid encoding a NeuroD1 polypeptide can be produced by techniques including, without limitation, molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a NeuroD1 polypeptide.

In some cases, a NeuroD1 polypeptide (e.g., a composition containing a NeuroD1 polypeptide) can be administered in addition to or in place of nucleic acid designed to express a NeuroD1 polypeptide. For example, a NeuroD1 polypeptide can be administered to a mammal to trigger reactive astrocytes within the brain into forming functional neurons.

Nucleic acid designed to express a NeuroD1 polypeptide (or a NeuroD1 polypeptide) can be delivered to glial cells (e.g., reactive astrocytes) within a brain (e.g., within the cerebral cortex) via direct intracranial administration, intrathecal administration, intraperitoneal administration, intravenous administration, intranasal administration, intramuscular administration, or oral administration in nanoparticles and/or drug tablets, capsules, or pills. Nucleic acid designed to express a NeuroD1 polypeptide (or a NeuroD1 polypeptide) can be delivered to glial cells (e.g., reactive astrocytes) within a brain (e.g., within the cerebral cortex) via any appropriate method (e.g., injection).

As described herein, nucleic acid designed to express a NeuroD1 polypeptide (e.g., a composition containing nucleic acid designed to express a NeuroD1 polypeptide) can be administered to a mammal (e.g., a human) having a brain injury to treat the brain injury. For example, an adeno-associated viral vector (e.g., a serotype 9 adeno-associated viral vector) can be designed to express a polypeptide having the amino acid sequence set forth in SEQ ID NO:1, and that designed viral vector can be administered to a human having a brain injury to treat the brain injury. In some cases, NeuroD1-mediated AtN conversion can treat brain injury by converting glial cells (e.g., reactive astrocytes) into functional neurons, rebalancing neuron:glia ratios, repairing damaged brain tissue (e.g., reversing glial scar tissue back to neural tissue), reducing neuroinflammation, restoring the blood-brain-barrier, and/or reducing the amount of toxic M1 microglia. In some cases, NeuroD1-mediated AtN conversion can take from about 7 to about 14 days after administration of nucleic acid designed to express a NeuroD1 polypeptide. In some cases, NeuroD1-mediated effects (e.g., converting reactive astrocytes into functional neurons, rebalancing neuron:glia ratios, repairing damaged brain tissue (e.g., reversing glial scar tissue back to neural tissue), reducing neuroinflammation, restoring the blood-brain-barrier, transforming A1 astrocytes, and/or reducing the amount of toxic M1 microglia) can be observed about 3 days after delivering nucleic acid encoding a NeuroD1 polypeptide or after delivering a composition containing a NeuroD1 polypeptide. In some cases, NeuroD1-mediated effects can be observed in reactive astrocytes prior to the reactive astrocytes being converting into neurons.

In some cases, the methods and materials provided herein (e.g., administering nucleic acid designed to express a polypeptide having the amino acid sequence set forth in SEQ ID NO:1 and/or administering a polypeptide having the amino acid sequence set forth in SEQ ID NO:1 to a mammal (e.g., a human) having a brain injury) can be used to convert glial cells (e.g., reactive astrocytes) into functional neurons. For example, from about 50% to about 95% (e.g., from about 50% to about 90%, from about 50% to about 85%, from about 50% to about 80%, from about 50% to about 75%, from about 50% to about 65%, from about 60% to about 95%, from about 70% to about 95%, from about 75% to about 95%, from about 80% to about 95%, from about 85% to about 95%, from about 90% to about 95%, from about 60% to about 90%, from about 65% to about 85%, or from about 70% to about 80%) of reactive astrocytes infected with a viral vector containing a nucleic acid sequence encoding a NeuroD1 polypeptide can undergo NeuroD1-mediated AtN conversion. In some cases, from about 85 to about 98 percent (e.g., 90.6±5.2%) of reactive astrocytes infected with a viral vector including a nucleic acid sequence encoding a NeuroD1 polypeptide can undergo NeuroD1-mediated AtN conversion. In some cases, from about 80 to about 98 percent (e.g., 89.2±4.7%) of reactive astrocytes infected with a viral vector including a nucleic acid sequence encoding a NeuroD1 polypeptide can undergo NeuroD1-mediated AtN conversion. For example, NeuroD1-mediated AtN conversion can form from about 200 functional neurons/mm² to about 800 functional neurons/mm² (e.g., from about 200 functional neurons/mm² to about 775 functional neurons/mm², from about 200 functional neurons/mm² to about 750 functional neurons/mm², from about 200 functional neurons/mm² to about 725 functional neurons/mm², from about 200 functional neurons/mm² to about 700 functional neurons/mm², from about 200 functional neurons/mm² to about 650 functional neurons/mm², from about 200 functional neurons/mm² to about 600 functional neurons/mm², from about 200 functional neurons/mm² to about 550 functional neurons/mm², from about 200 functional neurons/mm² to about 500 functional neurons/mm², from about 200 functional neurons/mm² to about 450 functional neurons/mm², from about 200 functional neurons/mm² to about 400 functional neurons/mm², from about 200 functional neurons/mm² to about 350 functional neurons/mm², from about 200 functional neurons/mm² to about 300 functional neurons/mm², from about 150 functional neurons/mm² to about 400 functional neurons/mm², from about 250 functional neurons/mm² to about 800 functional neurons/mm², from about 300 functional neurons/mm² to about 800 functional neurons/mm², from about 350 functional neurons/mm² to about 800 functional neurons/mm², from about 400 functional neurons/mm² to about 800 functional neurons/mm², from about 450 functional neurons/mm² to about 800 functional neurons/mm², from about 500 functional neurons/mm² to about 800 functional neurons/mm², from about 550 functional neurons/mm² to about 800 functional neurons/mm², from about 600 functional neurons/mm² to about 800 functional neurons/mm², from about 650 functional neurons/mm² to about 800 functional neurons/mm², from about 700 functional neurons/mm² to about 800 functional neurons/mm², from about 750 functional neurons/mm² to about 800 functional neurons/mm², from about 250 functional neurons/mm² to about 750 functional neurons/mm², from about 300 functional neurons/mm² to about 725 functional neurons/mm², from about 350 functional neurons/mm² to about 700 functional neurons/mm², from about 400 functional neurons/mm² to about 650 functional neurons/mm², from about 450 functional neurons/mm² to about 600 functional neurons/mm², or from about 500 functional neurons/mm² to about 550 functional neurons/mm²). In some cases, NeuroD1-mediated AtN conversion can form from about 150 to about 300 (e.g., 219.7±19.3) functional neurons/mm² after NeuroD1 infection. For example, NeuroD1-mediated AtN conversion can form functional neurons having increased expression of astrocytic genes that support neuronal functions (e.g., annexin A2 (Anax2), thrombospondin 1 (Thbs1), glypican 6 (Gpc6), and brain-derived neurotrophic factor (Bdnf) after NeuroD1 infection. In some cases, astrocytes are not depleted after NeuroD1-mediated AtN conversion. In some cases, astrocytes are repopulated (e.g., due to an intrinsic proliferation capability) following NeuroD1-mediated AtN conversion.

In some cases, methods and materials provided herein (e.g., administering nucleic acid designed to express a polypeptide having the amino acid sequence set forth in SEQ ID NO:1 (or a polypeptide having the amino acid sequence set forth in SEQ ID NO:1) to a mammal (e.g., a human) having a brain injury) can be used to rebalance neuron:glia ratios. Neuron:astrocyte ratios drop following brain injury (see, e.g., Example 1). In some cases, NeuroD1-mediated AtN conversion after NeuroD1 infection can increase the neuron:glia ratio in the brain of a mammal. For example, NeuroD1-mediated AtN conversion can increase the neuron:glia ratio by decreasing the number of astrocyes. For example, NeuroD1-mediated AtN conversion can increase the neuron:glia ratio by increasing the number of neurons. For example, NeuroD1-mediated AtN conversion can increase the neuron:glia ratio by both decreasing the number of astrocytes and increasing the number of neurons.

In some cases, the methods and materials provided herein (e.g., administering nucleic acid designed to express a polypeptide having the amino acid sequence set forth in SEQ ID NO:1 and/or administering a polypeptide having the amino acid sequence set forth in SEQ ID NO:1 to a mammal (e.g., a human) having a brain injury) can be used to repair damaged brain tissue (e.g., reversing glial scar tissue back to neural tissue). For example, NeuroD1-mediated AtN conversion can induce decreased expression of astrocytic genes unregulated in injury such as pan-reactive astrocyte genes (e.g., Gfap) and/or markers associated with glial scars (e.g., Lcn2 and CSPG) in the injured brain (e.g., relative to typical expression of the same astrocytic genes in non-injured brains). For example, NeuroD1-mediated AtN conversion can induce increased expression of astrocytic genes that support neuronal functions (e.g., Anax2, Thbs1, Gpc6, and Bdnf) in the injured brain (e.g., relative to typical expression of the same astrocytic genes in non-injured brains).

In some cases, the methods and materials provided herein (e.g., administering nucleic acid designed to express a polypeptide having the amino acid sequence set forth in SEQ ID NO:1 and/or administering a polypeptide having the amino acid sequence set forth in SEQ ID NO:1 to a mammal (e.g., a human) having a brain injury) can be used to reduce neuroinflammation. For example, NeuroD1-mediated AtN conversion can induce decreased expression of cytokines (e.g., TNFa and IL-1b) and/or markers for macrophages and monocytes (e.g., CD68) in the injured brain (e.g., relative to typical expression of the same cytokines in non-injured brains).

In some cases, the methods and materials provided herein (e.g., administering nucleic acid designed to express a polypeptide having the amino acid sequence set forth in SEQ ID NO:1 and/or administering a polypeptide having the amino acid sequence set forth in SEQ ID NO:1 to a mammal (e.g., a human) having a brain injury) can be used to restore the blood-brain-barrier. For example, NeuroD1-mediated AtN conversion can induce increased AQP4 expression (e.g., increased AQP4 signaling) in the injured brain along blood vessels (e.g., relative to typical expression of AQP4 along blood vessels in non-injured brains) in the brain of a mammal. For example, NeuroD1-mediated AtN conversion can induce increased AQP4 signaling between the injured brain and blood vessels in the brain of a mammal.

In some cases, the methods and materials provided herein (e.g., administering nucleic acid designed to express a polypeptide having the amino acid sequence set forth in SEQ ID NO:1 and/or administering a polypeptide having the amino acid sequence set forth in SEQ ID NO:1 to a mammal (e.g., a human) having a brain injury) can be used to transform A1 astrocytes (e.g., transform toxic A1 astrocytes into less harmful astrocytes). For example, NeuroD1-mediated AtN conversion can induce decreased expression of genes characteristic of A1 astrocytes (e.g., toxic A1 type astrocyte-specific genes such as Gbp2 and Serping1) in the injured brain (e.g., relative to typical expression of the same genes characteristic of A1 astrocytes in non-injured brains). For example, NeuroD1-mediated AtN conversion can induce decreased expression of Gbp2 and/or Serping1 in the injured brain.

In some cases, the methods and materials provided herein (e.g., administering nucleic acid designed to express a polypeptide having the amino acid sequence set forth in SEQ ID NO:1 and/or administering a polypeptide having the amino acid sequence set forth in SEQ ID NO:1 to a mammal (e.g., a human) having a brain injury) can be used to reduce microglia (e.g., to reduce the amount of toxic M1 microglia). For example, NeuroD1-mediated AtN conversion can form functional neurons having the morphology of resting microglia. For example, NeuroD1-mediated AtN conversion can reverse microglia morphology in the brain of a mammal (e.g., can reverse microglia morphology in the brain of a mammal back to the morphology of resting microglia).

In some cases, a polypeptide (or a nucleic acid encoding a polypeptide) containing the entire amino acid sequence set forth in SEQ ID NO:1, except that the amino acid sequence contains from one to ten (e.g., ten, one to nine, two to nine, one to eight, two to eight, one to seven, one to six, one to five, one to four, one to three, two, or one) amino acid additions, deletions, substitutions, or combinations thereof, can be used as described herein. For example, nucleic acid designed to express a polypeptide containing the entire amino acid sequence set forth in SEQ ID NO:1 with one to ten amino acid additions, deletions, substitutions, or combinations thereof can be designed and administered to a human having a brain injury to treat the brain injury.

Any appropriate amino acid residue set forth in SEQ ID NO:1 can be deleted, and any appropriate amino acid residue (e.g., any of the 20 conventional amino acid residues or any other type of amino acid such as ornithine or citrulline) can be added to or substituted within the sequence set forth in SEQ ID NO:1. The majority of naturally occurring amino acids are L-amino acids, and naturally occurring polypeptides are largely comprised of L-amino acids. D-amino acids are the enantiomers of L-amino acids. In some cases, a polypeptide provided herein can contain one or more D-amino acids. In some embodiments, a polypeptide can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions (e.g., one to ten amino acid substitutions) that can be used herein for SEQ ID NO:1 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine. Further examples of conservative substitutions that can be made at any appropriate position within SEQ ID NO:1 are set forth in Table 1.

TABLE 1
Examples of conservative amino acid substitutions.

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, polypeptides can be designed to include the amino acid sequence set forth in SEQ ID NO:1 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods disclosed herein.

In some cases, a polypeptide having an amino acid sequence with at least 85% (e.g., 85%, 86%, 87%, 88%, 89%, 90%, 91%, 92%, 93%, 94%, 95%, 96%, 97%, 98%, or 99.0%) sequence identity to the amino acid sequence set forth in SEQ ID NO:1, provided that it includes at least one difference (e.g., at least one amino acid addition, deletion, or substitution) with respect to SEQ ID NO:1, can be used. For example, nucleic acid designed to express a polypeptide containing an amino acid sequence with between 90% and 99% sequence identity to the amino acid sequence set forth in SEQ ID NO:1 can be administered to a human having a brain injury to treat the brain injury.

Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1) is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\Bl2seq c:\seq1.txt-j c:\seq2.txt-p blastn-o c:\output.txt-q−1-r2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt-j c:\seq2.txt-p blastp-o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:1), followed by multiplying the resulting value by 100. For example, an amino acid sequence that has 340 matches when aligned with the sequence set forth in SEQ ID NO:1 is 95.5 percent identical to the sequence set forth in SEQ ID NO:1 (i.e., 340±356×100=95.5056). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

In certain instances, the brain (e.g., the cerebral cortex) within a mammal (e.g., a living mammal) can be monitored to evaluate the effectiveness of a treatment described herein. Any appropriate method can be used to determine whether or not a brain injury present within a mammal is treated. For example, imaging techniques and/or laboratory assays can be used to assess the number reactive astrocytes and/or the number of functional neurons present within a mammal's brain. In some cases, imaging techniques and/or laboratory assays can be used to assess whether or not any NeuroD1-mediated effects (e.g., converting glial cells (e.g., reactive astrocytes) into functional neurons, rebalancing neuron:glia ratios, repairing damaged brain tissue (e.g., reversing glial scar tissue back to neural tissue), reducing neuroinflammation, restoring the blood-brain-barrier, transforming A1 astrocytes, and/or reducing the amount of toxic M1 microglia) are observed. In some cases, imaging techniques and/or laboratory assays can be used to assess NeuroD1-mediated effects can be as described in Example 1.

Also provided herein are kits that include using nucleic acid encoding a NeuroD1 polypeptide described herein (e.g., anti-cancer agents that inhibit IL-6, IL-8, and EGF). In some cases, the kits can include nucleic acid encoding a NeuroD1 polypeptide and nucleic acid encoding a recombinase (e.g., a cre recombinase). For example, the kits can include nucleic acid encoding a NeuroD1 polypeptide and nucleic acid encoding a recombinase located on the same vector (e.g., a viral vector). For example, the kits can include nucleic acid encoding a NeuroD1 polypeptide and nucleic acid encoding a recombinase (e.g., a cre recombinase) located on separate viral vectors. The kits also can include instructions for performing any of the methods described herein. In some cases, the kits can include at least one dose of any of the compositions (e.g., a composition containing nucleic acid encoding a NeuroD1 polypeptide and, optionally, nucleic acid encoding a recombinase) described herein. In some embodiments, the kits can provide a means (e.g., a syringe) for administering any of the compositions described herein.

The invention will be further described in the following examples, which do not limit the scope of the invention described in the claims.

### EXAMPLES

### Example 1: Reversing Glial Scar Back to Neural Tissue Through NeuroD1-Mediated Astrocyte-to-Neuron Conversion

This Example investigates in vivo glia-to-neuron conversion. A severe stab injury model in the mouse motor cortex was employed to investigate the impact of cell conversion on the microenvironment of injured brains. Different from the result of killing reactive astrocytes, converting reactive astrocytes into neurons reversed glial scar back to neural tissue. Astrocytes were not depleted after neuronal conversion, but rather repopulated after conversion. Ectopic expression of NeuroD1 in reactive astrocytes transformed A1 type toxic astrocytes into less reactive astrocytes. Reactive microglia were also ameliorated, and neuroinflammation was reduced following NeuroD1-mediated astrocyte-to-neuron (AtN) conversion. Furthermore, blood-brain-barrier (BBB) was restored, and neuronal synaptic connections were re-established after AtN conversion. Thus, the results provided herein demonstrate that NeuroD1-mediated cell conversion can reverse glial scar back to neural tissue by rebalancing neuron:glia ratio after injury.

### Methods and Materials

### Mouse Model of Stab Injury and Virus Injection

Animal procedures were performed in accordance with the Animal Protection Guidelines of the US National Institutes of Health, and all experimental protocols were approved by the Pennsylvania State University's Institutional Animal Care and Use Committee (IACUC). Wild type (WT) C57BL/6J and FVB/N-Tg(GFAP::GFP) 14Mes/J transgenic mice were purchased from Jackson Laboratory. Mice were housed in a 12-hour light/dark cycle and supplied with sufficient food and water. Adult mice (20-30 grams) with both genders were recruited in the experiments at the age of 3-6 months old. Mouse motor cortex was injured with a blunt needle (0.95 mm outer diameter) as described elsewhere (see, e.g., Bardehle et al., 2013 Nat Neurosci 16:580-586; Bush et al., 1999 Neuron 23:297-308; and Guo et al., 2014 Cell Stem Cell 14:188-202) with modifications. Briefly, ketamine/xylazine (100 mg/kg ketamine; 12 mg/kg xylazine) was administrated by intra-peritoneal injection. Under anesthesia, mice were placed in a stereotaxic apparatus with the skull and bregma exposed by a midline incision. A hole of about 1.2 mm was drilled in the skull in motor cortex (coordination: +1.0 mm anterior-posterior (AP), ±1.5 mm medial-lateral (ML) relative to Bergma). A blunt needle (0.95 mm) was placed into each site to the depth of −1.8 mm dorsal-ventral (DV) and stayed still for 3 minutes. At 4 or 10 days post stab injury (dps), mice were randomly subjected to either NeuroD1 or control virus-injection into the same site. The viral injection procedures were similar to those described elsewhere (see, e.g., Guo et al., 2014 Cell Stem Cell 14:188-202), with each injection site receiving 1.5 μL AAV or retrovirus using a 5 μL micro-syringe and a 34 Gauge needle (Hamilton). The viral injection rate was controlled at 0.15 μL/minute, with the needle gradually moved up at a speed of 0.1 mm/minute. After injection, the needle was maintained in place for additional 3 minutes before being fully withdrawn. Post-surgery, mice were recovered on heating pad until free movement was observed. Mice were single housed and carefully monitored daily for at least one week.

### AAV Vector Construction

The hGFAP promoter was obtained from pDRIVE-hGFAP plasmid (InvivoGen Inc.) and inserted into pAAV-MCS (Cell Biolab) between MluI and SacII to replace the CMV promoter. The Cre gene was obtained by PCR from Addgene plasmid #40591 (obtained from Dr. Albee Messing) and inserted into pAAV MCS between EcoRI and Sall sites to generate pAAV-hGFAP::Cre vector. To construct pAAV-FLEX-mCherry-P2A-mCherry and pAAV-FLEX-NeuroD1-P2A-mCherry vectors, the NeuroD1 or mCherry-coding cDNA was obtained by PCR using the retroviral constructs as described elsewhere (see, e.g., Guo et al., 2014 Cell Stem Cell 14:188-202). The NeuroD1 gene were fused with P2A-mCherry and subcloned into the pAAV-FLEX-GFP vector (Addgene plasmid #28304) between KpnI and XhoI sites. Plasmid constructs were verified by sequencing.

### AAV Virus Production

Recombinant AAV stereotype 9 was produced in 293AAV cells (Cell Biolabs). Briefly, triple plasmids (pAAV expression vector, pAAV9-RC (Cell Biolab), and pHelper (Cell Biolab)) were transfected by polyethylenimine (PEI, linear, MW 25,000). Cells were scrapped and centrifuged at 72 hours post transfection. Cell pellets were frozen and thawed for four times by being placed in dry ice/ethanol and 37° C. water bath alternately. AAV lysate was purified by ultra-centrifugation at 54,000 rpm for 1 hour in discontinuous iodixanol gradients (Beckman SW55Ti rotor). The virus-containing layer was collected followed by concentration by Millipore Amicon Ultra Centrifugal Filters. Virus titers were initially determined by QuickTiter™ AAV Quantitation Kit (Cell Biolabs): 1.2×10\hat{ }12 GC/mL for hGFAP::Cre, 1.4×10\hat{ }12 GC/mL for FLEX-NeuroD1-P2AmCherry, 1.6×10\hat{ }12 GC/mL for FLEX-mCherry-P2A-mCherry.

### Retrovirus Production

The pCAG::GFP-IRES-GFP retroviral vector was obtained. Mouse NeuroD1 sequence was inserted into the above-mentioned vector to generate pCAG::NeuroD1-IRES-GFP vector (Guo et al., 2014 Cell Stem Cell 14:188-202). To package retroviral particles, target vector with vesicular stomatitis virus glycoprotein (VSV-G) vector were transfected by PEI in gpg helper-free human embryonic kidney (HEK) cells. The titer of retroviral particles was determined as about 10⁷ particles/mL.

### Tissue Collection

Brain samples were collected as described elsewhere (see, e.g., Guo et al., 2014 Cell Stem Cell 14:188-202). Briefly, animals were injected with 2.5% Avertin for anesthesia. Transcardial perfusion with artificial cerebral spinal fluid (ACSF) was performed to systemically wash out the blood. Then, brains were dissected out, for immunohistochemistry use, brains were post-fixed in 4% paraformaldehyde (PFA) at 4° C. overnight. For RNA extraction use, brain tissues around the injury site (about 1.5×1.5 mm square) were dissected and flash-frozen at −80° C.

### Immunohistochemistry

After fixation, brain tissues were sectioned at 40 μm sections using Leica-1000 vibratome. Brain slices were washed 3 times with phosphate-buffered saline (PBS) followed by permeablization in 2% Triton X-100 in PBS for 1 hour. Then, brain sections were blocked in 5% normal donkey serum and 0.3% Triton X-100 in PBS for 1 hour. The primary antibodies were added into blocking buffer and incubated with brain sections for overnight at 4° C. Primary antibodies were rinsed off with PBS for 3 times followed by secondary antibody incubation for 2 hours at room temperature (RT). After being washed with PBS, brain sections were mounted onto a glass slide with an anti-fading mounting solution containing DAPI (Invitrogen). Images were acquired with confocal microscopes (Olympus FV1000 or Zeiss LSM800). To ensure antibody specificity, only secondary antibody was used for immunostaining as a side-by-side control, with no distinct signal detected.

### BrdU Labeling

For labeling of proliferative astrocytes after brain injury, GFAP-GFP transgenic mice were used and intra-peritoneal injection of BrdU (BrdU labeling reagent, Invitrogen) was conducted daily from 1 dps to 4 or 10 dps at a dose of 0.1 mL/10 grams. For characterization of newly generated astrocytes after conversion, BrdU was administrated daily from 7 to 14 dpi. Fixed brain sections were subjected to a 30-minute treatment with 2 M HCl at 37° C. for DNA denaturation. After 5 washes with PBS, brain sections were permeablized in 2% Triton-PBS for 1 hour and incubated in blocking buffer (5% normal donkey serum and 0.3% triton in PBS) for additional 1 hour at room temperature. Primary antibodies were mixed in blocking solution and incubated with brain sections at 4° C. overnight.

### Cresyl Violet Staining (Niss1 Staining)

A series of coronal sections were collected for analysis of tissue damage. The center of needle injury was collected and set as zero point; two sections anterior and posterior to the injury site at 200 μm intervals also were selected. The brain sections were first placed in xylene for 5 minutes, followed by a gradual hydration series with alcohol (95%, 70%, and 0% in water) for 3 minutes each. The samples were transferred to cresyl violate buffer (0.121 mg/mL cresyl violet acetate in NaAc buffer, pH 3.5) for 8 minutes at 60° C. Upon completion, the stain was rinsed and dehydrated in a series with alcohol (0%, 70%, 95%, and 100%) for 30 seconds each. Finally, the samples were cleared in xylene for 1 hour and mounted in DPX Mountant (Sigma-Aldrich). Images were acquired using Olympus BX61 microscope.

### RNA Extraction and Quantitative Real-Time PCR

The RNA extraction was performed using Macherey-Nagel NucleoSpin RNA kit. RNA concentration and purity were measured by NanoDrop. For cDNA synthesis, 500 ng RNAs were mixed with Quanta Biosciences qScript cDNA supermix and incubated at 25° C. for 5 minutes, 42° C. for 30 minutes, 85° C. for 5 minutes, and held at 4° C. Upon completion, the cDNAs were diluted 5-fold with RNase/DNase-free water. The primers for real-time qPCR were designed using Applied Biosystems Primer Express software and synthesized in IDT. For qRT-PCR each reaction, 6.25 μL, SYBR Green Supermix (Quanta Biosciences PerfeCTa, ROX), 2 μL cDNA, and 3.75 μL water were mixed well and loaded in 96-well plate (Applied Biosystem Inc). Gapdh was used as an internal control. All comparisons were conducted to non-injured cortical tissue from healthy mice. Comparative Ct method was used for calculation of fold change.

### BBB Permeability Test

Mice were anesthetized and perfused with ACSF as described above, followed by 15 mL of 0.5 mg/mL Sulfo-NHS-LC-Biotin in PBS. For immunohistochemistry, after primary antibody incubation, brain sections were incubated with FITC Streptavidin, 1:800 diluted in PBS+0.3% triton+2.5% normal goat or donkey serum at room temperature for 1 hour, followed by normal mounting procedures.

### Electrophysiology

Brain slice recordings were performed as described elsewhere (see, e.g., Guo et al., 2014 Cell Stem Cell 14:188-202). Briefly, 1 month after NeuroD1-AAV injection, mice were anaesthetized with 2.5% avertin, and then perfused with NMDG-based cutting solution (in mM): 93 NMDG, 93 HCl, 2.5 KCl, 1.25 NaH₂PO₄, 30 NaHCO³, 20 HEPES, 15 glucose, 12 N-Acetyl-L-cysteine, 5 sodium ascorbate, 2 Thiourea, 3 sodium pyruvate, 7 MgSO₄, 0.5 CaCl₂, pH 7.3-7.4, 300 mOsmo, bubbled with 95% O₂/5% CO₂. Coronal sections of 300 μm thickness were cut around AAV-injected cortical areas with a vibratome (VT1200S, Leica, Germany) at room temperature. Slices were collected and incubated at 33.0±1.0° C. in oxygenated NMDG cutting solution for 10-15 minutes. Then, slices were transferred to holding solutions with continuous 95% O₂/5% CO₂ bubbling (in mM): 92 NaCl, 2.5 KCl, 1.25 NaH₂PO₄, 30 NaHCO₃, 20 HEPES, 15 glucose, 12 NAcetyl-L-cysteine, 5 sodium ascorbate, 2 Thiourea, 3 sodium pyruvate, 2 MgSO₄, and 2 CaCl₂. Brain sections were recovered in the holding solution at least for 0.5 hours at room temperature. For patch clamp recording, a single slice was transferred to the recording chamber continuously perfused with standard ACSF (in mM: 124 NaCl, 2.5 KCl, 1.25 NaH₂PO₄, 26 NaHCO₃, 10 Glucose, 1.3 MgSO₄, and 2.5 CaCl₂) saturated by 95% O₂/5% CO₂ at 33.0±1.0° C. To record action potentials and ionic currents, whole-cell recordings were performed with pipette solution containing (in mM): 135 K-Gluconate, 10 KCl, 5 Na-phosphocreatine, 10 HEPES, 2 EGTA, 4 MgATP, and 0.3 Nae GTP, pH 7.3 adjusted with KOH, 280-290 mOsm. Depolarizing currents were injected to elicit action potentials under current-clamp model. To record spontaneous excitatory postsynaptic currents (sEPSCs) and spontaneous inhibitory postsynaptic currents (sIPSCs), pipette solution contained (in mM): 120 Cs-Methanesulfonate, 10 KCl, 10 Na-phosphocreatine, 10 HEPES, 5 QX-314, 1 EGTA, 4 MgATP, and 0.3 Na₂GTP, pH 7.3 adjusted with KOH, 280-290 mOsm. The cell membrane potentials were held at −70 mV (the reversal potential of ionotropic glutamate receptors) for sEPSC recording, and 0 mV (the reversal potential of GABAA receptors) for sIPSC recording, respectively. Data were collected with a MultiClamp 700A amplifier and analyzed with pCLAMP10 software (Molecular Devices).

### Data Analysis and Statistics

Prism 6 graphpad software was used for statistical analysis and bar graphs. For comparison of two data sets, Student's t-test was conducted. For comparison of three data sets, one-way or two-way analysis of variance (ANOVA) was performed, followed by post-hoc tests. Statistical significance was set as p<0.05. Data were presented as mean±SEM.

### Antibodies and Primers

The antibodies used in this study are set forth in Table 2, and the primers used in this study are set forth in Table 3.

TABLE 2
Primary antibodies used in the study

| Primary | | | | |
|---|---|---|---|---|
| antibody | Species | Company | Catalog# | Dilution |
| AQP4 | Rabbit | Santa Cruz | Sc-20812 | 1:600 |
| BrdU | Rat | Accurate Chemical | YSRTMCA | 1:600 |
| | | | 2060GA | |
| CD68 | Mouse | Abcam | Ab31630 | 1:600 |
| Connexin43 | Rabbit | Abcam | Ab11730 | 1:800 |
| CSPG | Mouse | Sigma | C8035 | 1:600 |
| GFP | Chicken | Abcam | Ab13970 | 1:1000 |
| GFAP | Rabbit | Millipore | AB5804 | 1:1000 |
| GFAP | Chicken | Millipore | AB5541 | 1:1000 |
| Iba1 | Rabbit | Wako | 019-19741 | 1:800 |
| Iba1 | Goat | Abcam | Ab5076 | 1:400 |
| iNOS | Mouse | Millipore | MABN533 | 1:400 |
| LCN2 | Gt | R&D | AF1857 | 1:600 |
| LY6C | Rat | Abcam | AB15627 | 1:800 |
| MAP2 | Chicken | Abcam | AB5392 | 1:400 |
| MBP | Chicken | Millipore | AB9348 | 1:400 |
| NeuN | Rabbit | Millipore | ABN78 | 1:1000 |
| NeuroD1 | Mouse | Abcam | AB60704 | 1:600 |
| NG2 | Mouse | Millipore | MAB5384 | 1:400 |
| RFP | Rat | Antibodies-online.com | ABIN334653 | 1:1000 |
| S100b | Mouse | Abcam | Ab66028 | 1:800 |
| SMI32 | Mouse | Biolegend | 801701 | 1:1000 |
| SMI312 | Mouse | Biolegend | SMI312R | 1:1000 |
| SV2 | Mouse | DSHB | SV2 | 1:1000 |
| vGlut1 | Rabbit | Synaptic system | | 1:1000 |
| GAD67 | Mouse | Millipore | MAB5406 | 1:1000 |

TABLE 3
Primers used in the study

| | | Seq ID | | Seq ID |
|---|---|---|---|---|
| Primer | Forward sequence | No | Reverse sequence | No |
| m-Gapdh | GGAGCGAGACCCCACTAACA | 2 | ACATACTCAGCACCGGCCTC | 3 |
| m-NeuroD1 | AAAGCCCCCTAACTGACTGCA | 4 | TCAAACTCGGCGGATGGTT | 5 |
| m-Gfap | TTCAGCCACACCTTTCCAGC | 6 | CCTTAGAGGAGGCCTGGGAG | 7 |
| m-Lcn2 | AACTTGATCCCTGCCCCATCT | 8 | TTTCTGGACCGCATTGCCT | 9 |
| m-Gbp2 | GGGGTCACTGTCTGACCACT | 10 | GGGAAACCTGGGATGAGATT | 11 |
| m-Serpingl | ACAGCCCCCTCTGAATTCTT | 12 | GGATGCTCTCCAAGTTGCTC | 13 |
| m-Anax2 | CAGGACATTGCCTTCGCCTAT | 14 | TAGGCCCAAAATCACCGTCTC | 15 |
| m-Thbsl | CGTGAGCGATGAGAAGGACA | 16 | CGATCTGTGCTTGGTTGTGC | 17 |
| m-Gpc6 | CGGCCAGACACTTTCATCAGA | 18 | TGGATTCATCGCTTGTGTCTTG | 19 |
| m-S100a10 | CCTCTGGCTGTGGACAAAAT | 20 | CTGCTCACAAGAAGCAGTGG | 21 |
| m-Tm4sf1 | GCCCAAGCATATTGTGGAGT | 22 | AGGGTAGGATGTGGCACAAG | 23 |
| m-Il1b | TTGAAGTTGACGGACCCCAA | 24 | TGTTGATGTGCTGCTGCGA | 25 |
| m-Il6 | TTCCATCCAGTTGCCTTCTTG | 26 | CATTTCCACGATTTCCCAGAG | 27 |
| m-Tnf | CACAAACCACCAAGTGGAGGA | 28 | ACAAGGTACAACCCATCGGCT | 29 |

### Results

### High Efficiency of NeuroD1-Mediated Astrocyte-to-Neuron Conversion in a Severe Stab Injury Model

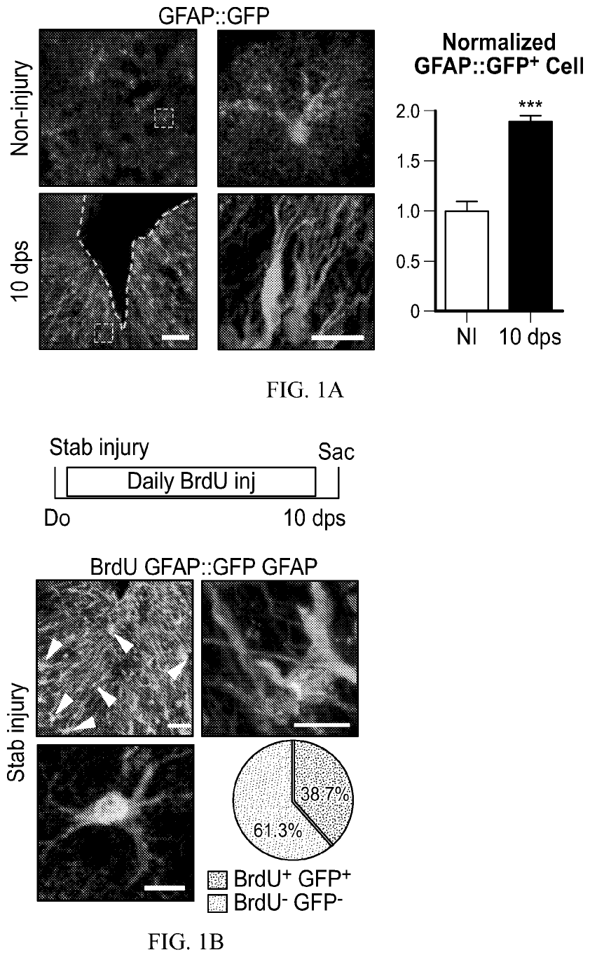

Reactive glial cells can be directly converted into functional neurons inside mouse brains by a single transcription factor NeuroD1 (Guo et al., 2014 Cell Stem Cell 14:188-202). To determine if glial scar aids axon regeneration, and if converting astrocytes into functional neurons results in any detrimental effects, a severe stab injury model was established in adult mice (3-6 months old, both gender included) and investigated the impact of NeuroD1-mediated AtN conversion on the microenvironment of the injury areas. Specifically, a blunt needle (outer diameter 0.95 mm) was used to make a severe stab injury in the mouse motor cortex, which induced a significant tissue loss together with reactive astrogliosis in the injury sites (FIG. 1a). As expected, the number of astrocytes increased significantly in the stab-injured areas at 10 days post stab injury (dps) (FIG. 1a, right bar graph). This is consistent with the intrinsic proliferative capability of astrocytes following neural injury, as shown by bromodeoxyuridine (BrdU) incorporation during cell division (FIG. 1b; BrdU+ astrocytes, 38.7±2.5%, n=4 mice, 10 dps).

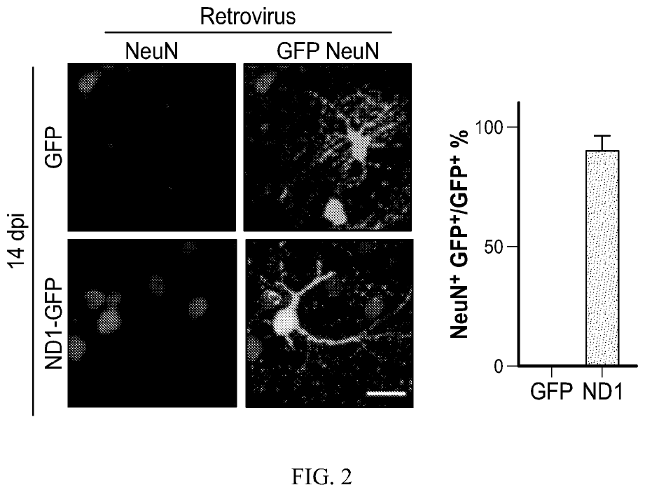

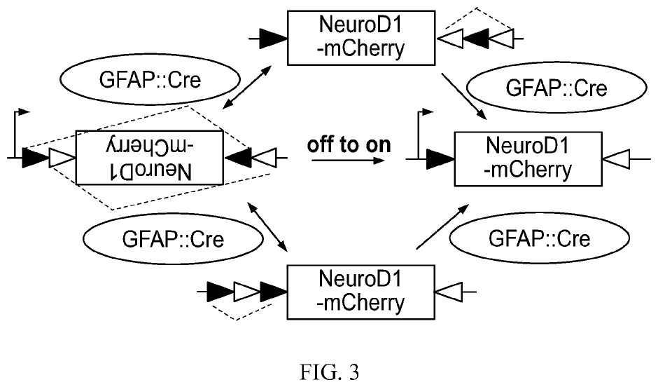

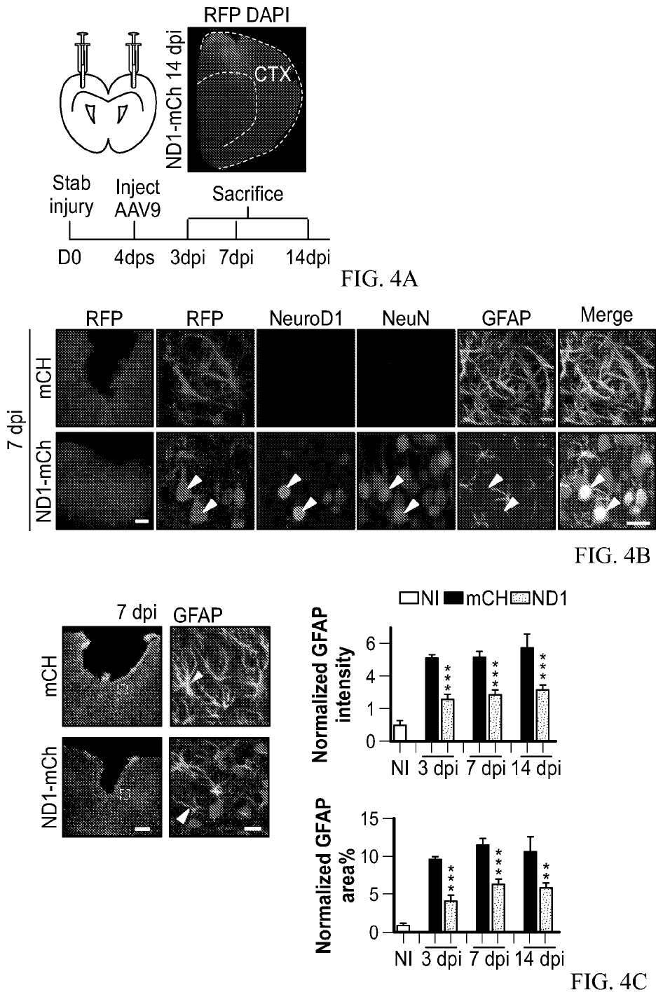

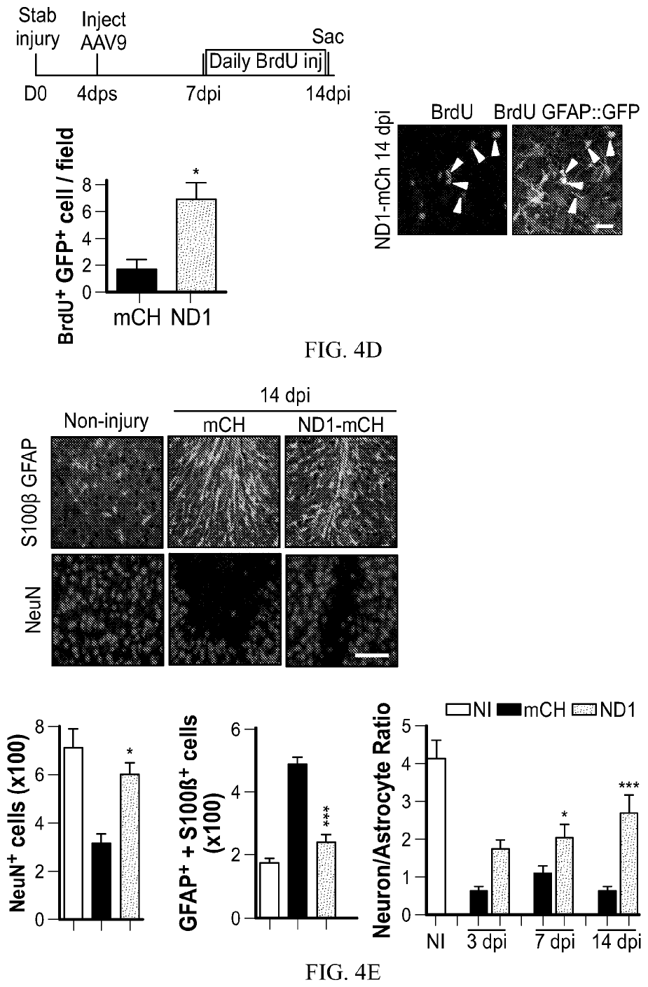

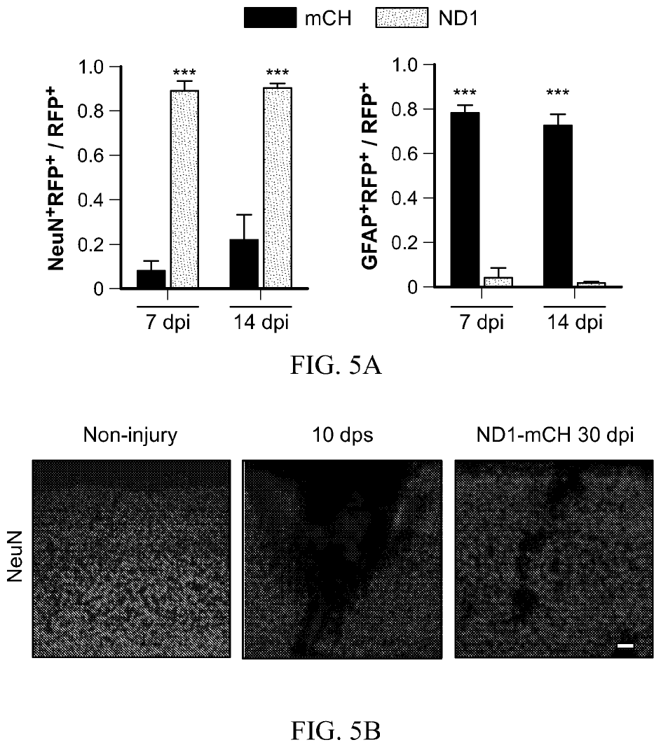

To convert glial cells into neurons, retroviruses expressing NeuroD1 were employed in dividing reactive glial cells as described elsewhere (see, e.g., Guo et al., 2014 Cell Stem Cell 14:188-202). Efficient (90.6±5.2%) ectopic expression of NeuroD1-GFP in glial cells converted them into NeuN⁺ neurons, whereas none of the GFP-infected cells were co-labeled by NeuN in the control group (FIG. 2, n=4 mice). Despite high conversion efficiency, the total number of newly converted neurons after retroviral infection was limited due to the limited number of glial cells that happened to be dividing during retroviral injecting. To increase the total number of newly converted neurons in the injury sites for therapeutic repair, an AAV Cre-FLEX system was developed to achieve broader viral infection and cell conversion because AAV can express target genes in both dividing and non-dividing cells (Ojala et al., 2015 Neuroscientist 21:84-98). Cre recombinase was expressed under the control of astrocyte promoter GFAP (GFAP::Cre) to specifically target astrocytes. Cre acted at the loxP-type recombination sites flanking an inverted sequence of NeuroD1-P2A-mCherry under the CAG promoter in a separate AAV vector (FLEX-CAG::NeuroD1-P2A-mCherry) (FIG. 3). Therefore, NeuroD1 expression targeted to reactive astrocytes where GFAP promoter is highly active, and the subsequent Cre-mediated recombination led to high expression of NeuroD1 driven by a strong promoter CAG. This AAV Cre-FLEX system was very efficient as shown by wide expression of NeuroD1-mCherry in the stab-injured cortical areas (FIG. 4a). At 7 days post viral injection (dpi) (injected at 4 dps), the control mCherry AAV-infected cells were mostly GFAP+ astrocytes, whereas the majority of NeuroD1-mCherry infected cells had become NeuN⁺ neurons (FIG. 4b). NeuroD1-mediated astrocyte-to-neuron conversion efficiency was 89.2±4.7% (7 dpi, n=4 mice), whereas in control group 80% of mCherry AAV-infected cells were astrocytes (FIG. 5a). The number of NeuroD1-converted neurons in the injury areas were quantified as 219.7±19.3/mm² at 14 dpi. Together, by developing an AAV Cre-FLEX system to highly express NeuroD1 in reactive astrocytes, high efficiency of AtN conversion was achieved in stab-injured mouse cortex.

Astrocytes not Depleted after Conversion

To determine whether the high efficiency of AtN conversion resulted in depletion of astrocytes after neuronal conversion, GFAP staining was performed, and an overall reduction of GFAP signal in the NeuroD1 group compared to the control group was observed (FIG. 4c). Unexpectedly, not only the GFAP signal was reduced, but also the astrocyte morphology showed a significant change in the NeuroD1 group, displaying much less hypertrophic processes compared to the control group (FIG. 4c, arrowhead in left images), suggesting that astrocytes in the NeuroD1-converted areas became less reactive. Quantitative analysis found that the GFAP signal was significantly increased by 5-10 fold after stab injury in the control group (FIG. 4c, black bar), but reduced significantly by half in NeuroD1-infected areas (FIG. 4c, gray bar). The decrease of GFAP signal in NeuroD1 group was consistent with the conversion of reactive astrocytes into neurons. On the other hand, detection of a significant level of GFAP signal in the NeuroD1-infected areas suggested that reactive astrocytes were not depleted after conversion. Since astrocytes have intrinsic capability to proliferate, whether neuronal conversion might trigger the remaining astrocytes to proliferate was examined. To test this idea, BrdU, which can be incorporated into DNA during cell division, was injected daily from 7 dpi (viral injection at 4 dps) to 14 dpi in order to monitor cell proliferation in both control group and NeuroD1 group (FIG. 4d, left schematic illustration). Interestingly, it was discovered that the number of BrdU-labeled astrocytes in the NeuroD1 group more than tripled that of the control group (FIG. 4d, right panels and bar graph for quantification). Many of the BrdU-labeled astrocytes were adjacent to the NeuroD1-converted neurons (FIG. 4d, arrow head), suggesting that astrocytes can self-regenerate following AtN conversion. Therefore, astrocytes were not depleted by AtN conversion, but rather were repopulated.

Neuron:Astrocyte Ratio Rebalanced after Conversion

Brain functions rely upon a delicate balance between neurons and glial cells. After neural injury, neurons die but glial cells proliferate, leading to an altered neuron:glia ratio in the injury areas. This was clearly reflected in the severe stab injury model, where the number of healthy neurons (NeuN⁺ cells) significantly decreased after injury but the number of astrocytes significantly increased (GFAP⁺/s100b⁺) (FIG. 4e). Interestingly, after NeuroD1-mediated AtN conversion, the NeuN⁺ neurons significantly increased but the number of astrocytes decreased (FIG. 4e), a clear reversal from the injury. Quantitative analysis discovered that the neuron:astrocyte ratio in the mouse motor cortex was about 4:1 (4 neurons to one astrocyte) in resting condition (FIG. 4e, white bar in the right bar graph). After stab injury, the neuron:astrocyte ratio dropped to <1 (FIG. 4e, black bar). After NeuroD1-conversion, the neuron:astrocyte ratio reversed back to 2.6 at 14 dpi (FIG. 4e, gray bar). Such significant reversal of the neuron:astrocyte ratio can be involved in functional recovery in injured brains.

### A1 Reactive Astrocytes Transformed During Conversion

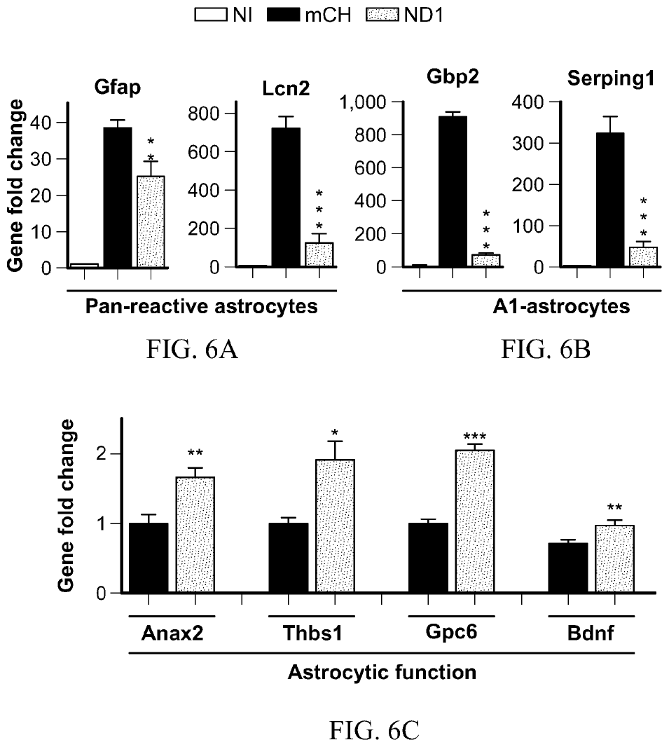

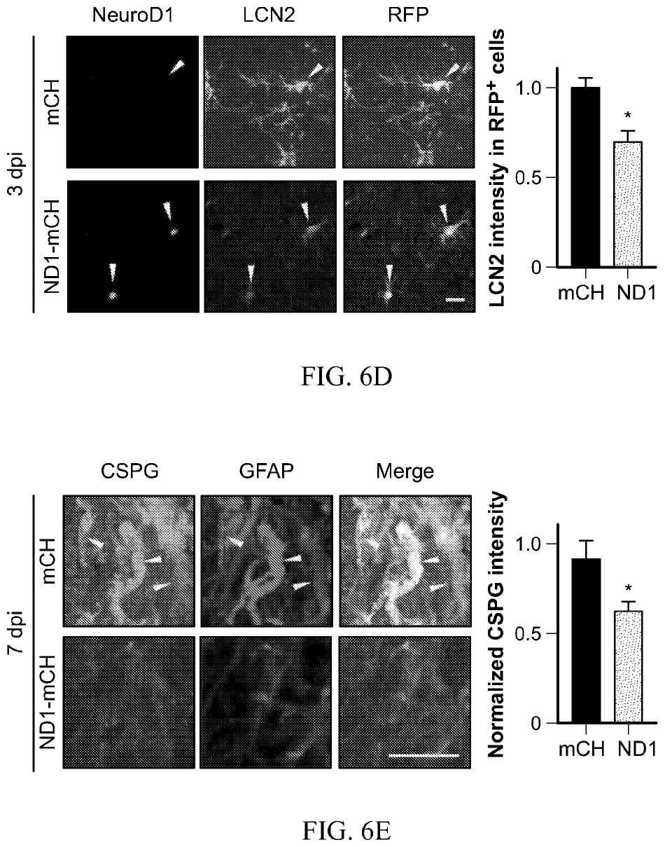

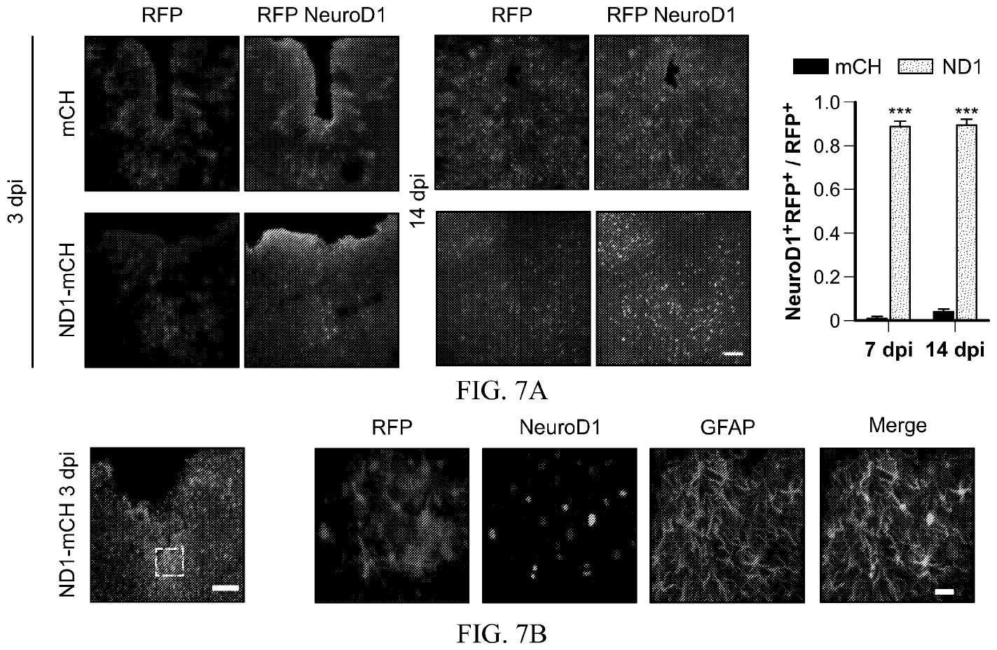

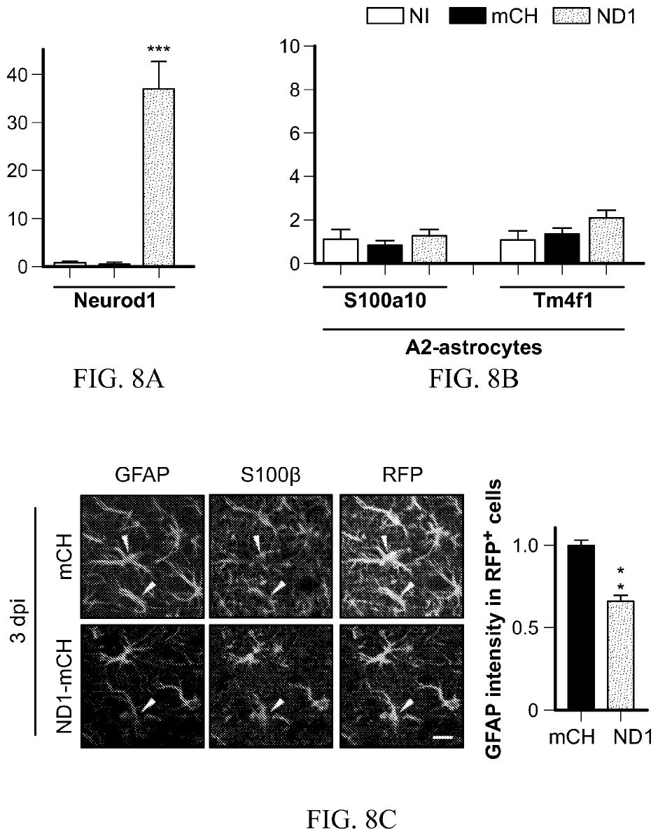

To determine if astrocytes persisted after NeuroD1-mediated conversion, and to evaluate whether they differ from the reactive astrocytes in the control group, RT-PCR analysis of a variety of genes related to A1 astrocytes and neuroinflammation at 3 dpi, an early stage before neuronal conversion, was performed. Compared to non-injury (NI) group, stab injury caused an upregulation of the pan-reactive astrocyte genes such as Gfap by 37-fold (FIG. 6a), which was significantly attenuated in the NeuroD1 group (One-way ANOVA, Sidak's test, **P<0.01, n=4 pairs). Lcn2, a neuroinflammation marker associated with reactive astrocytes after injury, was increased by 700-fold after stab injury, but drastically reduced in NeuroD1 group (FIG. 6a, One-way ANOVA, Sidak's test, ***P<0.001, n=4 pairs). In addition, genes characteristic for A1 astrocytes such as Gbp2 and Serping1 were upregulated by 300-900 folds after stab injury (One-way ANOVA, Sidak's test, ***P<0.001, n=4 pairs), but greatly attenuated after NeuroD1 treatment (FIG. 6b). This remarkable change was unexpected, because at 3 dpi after NeuroD1 infection, astrocytes have not been fully converted into neurons yet (FIG. 7a,b). Immunostaining at 3 dpi confirmed that NeuroD1 was indeed expressed in infected astrocytes (FIG. 7b; 87.4±2.5% mCherry⁺ cells were NeuroD1⁺; 92.8±2.8% mCherry⁺ cells were GFAP⁺, n=6 mice). Nevertheless, A1 astrocytes were already inhibited or transformed before neuronal conversion. In addition, NeuroD1 treatment appeared to increase the astrocytic genes that support neuronal functions such as Anax2, Thbs1, Gpc6, and Bdnf (FIG. 6c). As a validation of the RT-PCR results, NeuroD1 overexpression was confirmed by RT-PCR analysis (FIG. 8a). A2 astrocyte genes were not changed despite a decrease of GFAP expression after NeuroD1-infection (FIG. 8b,c).

NeuroD1-positive astrocytes also exhibited a significant decrease in the expression level of Lcn2 compared to the reactive astrocytes in control group (FIG. 6d, Student's t test, *P<0.05, n=3 pairs), consistent with the RT-PCR analysis in FIG. 6a. Furthermore, CSPG was widely associated with reactive astrocytes after neural injury and played a role in neuroinhibition during glial scar formation. In the severe stab injury model, a high level of CSPG was detected in the injury areas (FIG. 6e, top row); but in NeuroD1-treated group, the CSPG level was significantly reduced (FIG. 6e, bottom row; quantified in bar graph, Student's t test, *P<0.05, n=5 pairs). Together, these results suggest that ectopic expression of NeuroD1 in reactive astrocytes significantly attenuated their reactive and neuroinflammatory properties. Importantly, such beneficial effects occurred as early as 3 days after NeuroD1 infection, even before astrocytes were converting into neurons.

### Astrocyte-Microglia Interaction During Neuronal Conversion

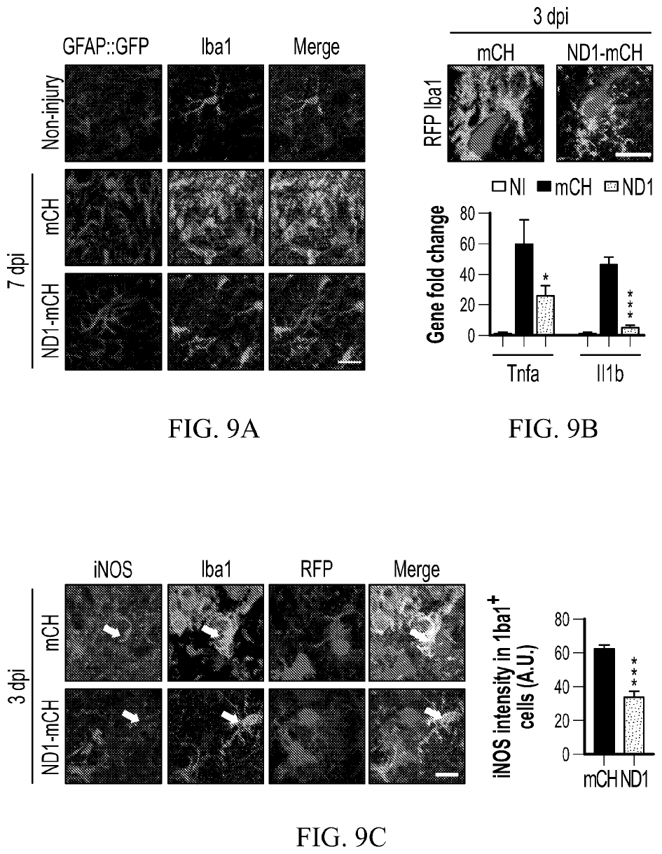

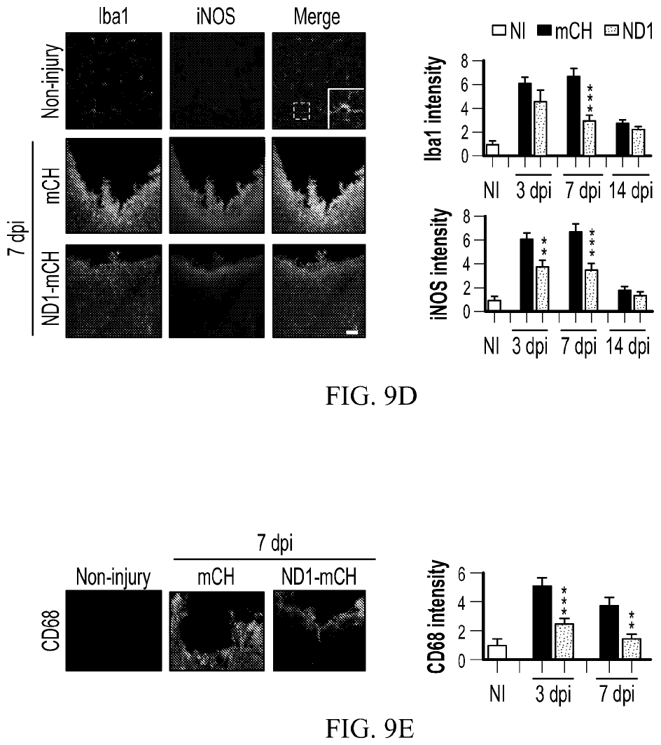

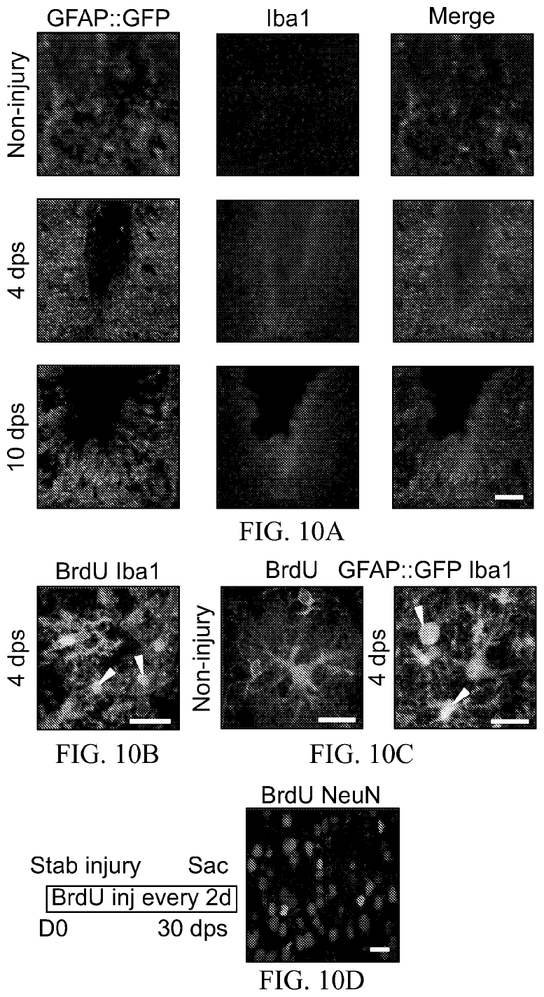

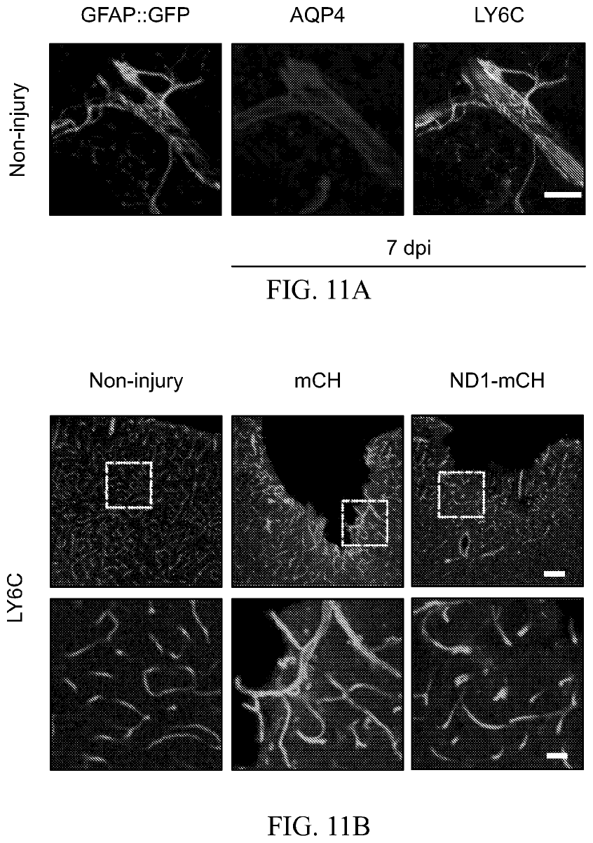

Next, the impact of AtN conversion on microglia was investigated. Compared to the resting microglia in non-injured brains (FIG. 9a, top row), stab injury induced reactive microglia were hypertrophic and amoeboid-shape (FIG. 9a, middle row). Both microglia and astrocytes were highly proliferative after stab injury as shown by BrdU labeling (FIG. 10a-c), but no newborn neurons were detected in the adult mouse cortex after stab injury (FIG. 10d). In NeuroD1-infected areas, however, microglia morphology reversed back and was closer to the resting microglia with ramified processes (FIG. 9a, bottom row). Such morphological change started as early as 3 dpi (FIG. 9b), where microglia contacting NeuroD1-infected astrocytes were much less reactive compared to the microglia contacting mCherry-infected astrocytes. RT-PCR analysis revealed that the cytokines TNFα and IL-1b were both significantly increased after stab injury, but both attenuated in the NeuroD1 group (FIG. 9b, bar graph, One-way ANOVA followed by Turkey's test, *P<0.05, ***P<0.001, n=4 pairs). Such dramatic decrease of cytokines during AtN conversion may explain why microglia were less reactive in the NeuroD1 group. Consistently, toxic M1 microglia that were immunopositive for iNOS exhibited a significant reduction in the NeuroD1 group compared to the control group (FIG. 9c, Student's t test, ***P<0.001, n=4 pairs). The reduction of iNOS-labeled M1 microglia coincided with the reduction of toxic A1 astrocytes, as detected at 3 dpi after NeuroD1 infection, suggesting an intimate interaction between astrocytes and microglia. At 7 dpi, compared to the control group, the reduction of iNOS in the NeuroD1 group was even more significant, accompanied with a reduction of Iba1 signal as well (FIG. 9d, two-way ANOVA, **P<0.01, ***P<0.001, n=6 pairs at 3 dpi and 7 dpi, n=5 pairs at 14 dpi). In addition, stab injury induced a remarkable increase in the expression level of CD68, a marker for macrophages and monocytes as well as some reactive microglia (FIG. 9e). However, in NeuroD1-infected areas, the expression level of CD68 was significantly attenuated (FIG. 9e, bar graph, two-way ANOVA, **P<0.01, ***P<0.001, n=6 pairs). Together, these results suggest that accompanying astrocyte-to-neuron conversion, toxic M1 microglia were reduced and neuroinflammation was alleviated.

### Astrocyte-Blood Vessel Interaction During In Vivo Cell Conversion

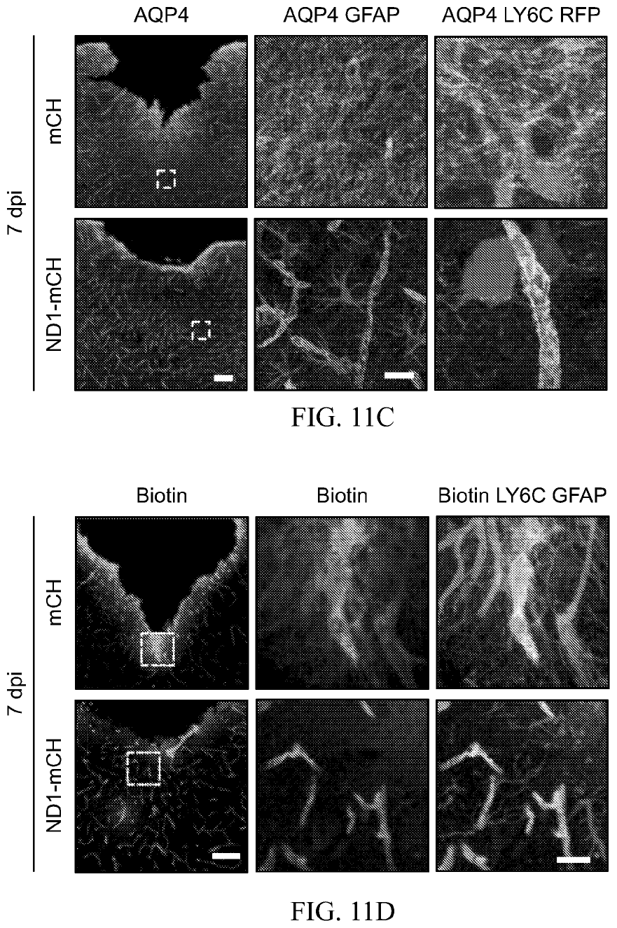

A function of astrocytes in the brain is to interact with blood vessels and contribute to blood-brain-barrier (BBB) in order to prevent bacterial and viral infection and reduce chemical toxicity (Obermeier et al., 2013 Nat Med 19:1584-1596). In healthy brain, BBB is tightened by astrocytic endfeet wrapping around the blood vessels (FIG. 11a). Comparing to the evenly distributed blood vessels (labeled by endothelial marker Ly6C) in non-injured brains (FIG. 11b, left panels), stab injury caused blood vessels to become swollen (FIG. 11b, middle panels). In NeuroD1-treated group, however, blood vessels exhibited less hypertrophic morphology and closer to the ones in healthy brains (FIG. 11b, right panels). Accompanying altered blood vessel morphology after stab injury was a disruption of BBB, as evident by the mislocalization of AQP4 signal. AQP4 is a water channel protein, normally concentrating at the endfeet of astrocytes at resting state wrapping around blood vessels (see FIG. 11a). After stab injury, the AQP4 signal dissociated from blood vessels and instead distributed throughout the injury areas (FIG. 11c, top row). In NeuroD1-treated areas, AQP4 signal exhibited reassociation with blood vessels, returning back to a normal state (FIG. 11c, bottom row). The astrocytic morphology also looked much less reactive in NeuroD1-treated group (FIG. 11c, bottom row). To further evaluate BBB integrity, the mice were perfused with biotin, a molecule that can easily leak out after BBB breakdown. After stab injury, a significant leakage of biotin in the injured areas was observed in control group expressing mCherry alone (FIG. 11d, top row). However, in NeuroD1 group, biotin was mainly detected inside the blood vessels, and the leakage was significantly reduced in the parenchyma tissue (FIG. 11d, bottom row), indicating restoration of BBB integrity. Together, these results suggest that after NeuroD1-mediated cell conversion, astrocytes interact with blood vessels again to restore the broken BBB caused by neural injury.

Functional Recovery after NeuroD1-Mediated Neuronal Conversion

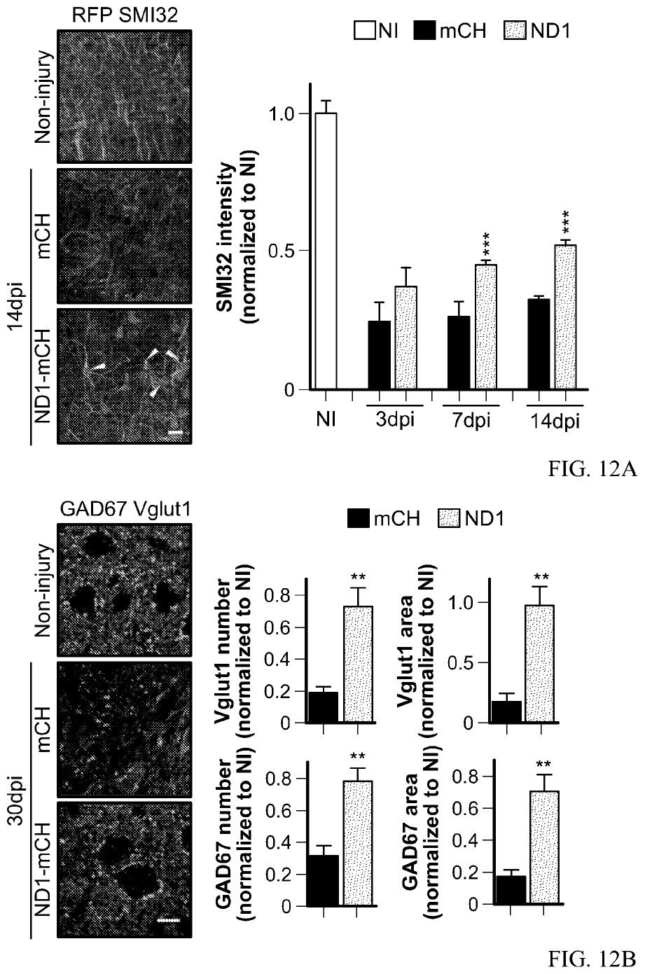

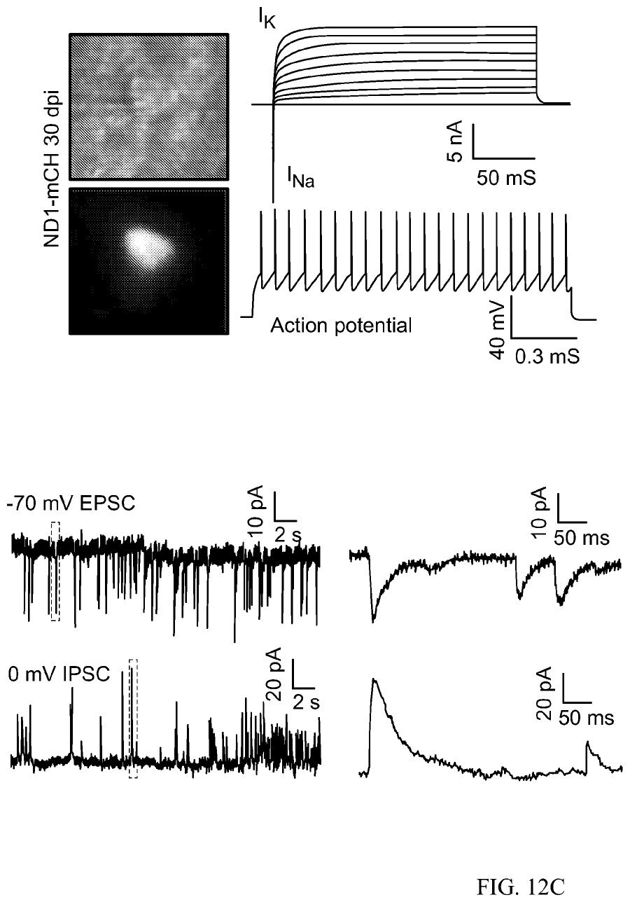

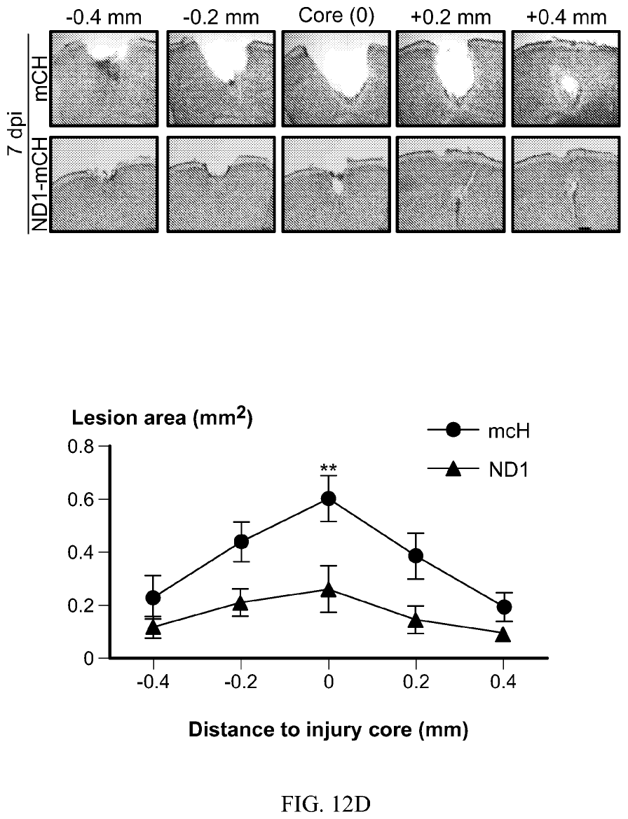

With a substantial change in the glial environment after NeuroD1-mediated AtN conversion, neuronal properties such as dendritic morphology, synaptic density, and electrophysiological function in the injury areas were investigated. Stab injury resulted in severe dendritic damage as shown by dendritic marker SMI32 (FIG. 12a, left images, green signal). In NeuroD1-infected areas, however, a significant increase in dendritic signal SMI32 was detected (FIG. 12a, bottom image). Quantitatively, neuronal dendrites labeled by SMI32 were severely injured after stab lesion, decreasing to 25% of the non-injured level, but NeuroD1 treatment rescued the dendritic signal to over 50% of the non-injured level at 14 dpi (FIG. 12a; bar graph, two-way ANOVA, ***P<0.001, n=4 pairs). Consistent with the dendritic damage, stab injury also caused a severe reduction of synapses in the injured areas as shown by glutamatergic synapse marker vGluT1 and GABAergic synapse marker GAD67 (FIG. 12b, left images). After NeuroD1 treatment (30 dpi), both glutamatergic and GABAergic synaptic density in the injured areas exhibited a significant increase compared to the control group (FIG. 12b, bar graphs).

It was next investigated whether the NeuroD1-converted neurons were physiologically functional. Cortical brain slices were prepared, and patch-clamp recordings were performed on the NeuroD1-mCherry converted neurons (FIG. 12c, left images). After one month of conversion, the NeuroD1-mCherry positive neurons exhibited large sodium and potassium currents (>5 nA) and repetitive action potentials (FIG. 12c, left traces). More importantly, both glutamatergic synaptic events (frequency: 0.96±0.5 Hz; amplitude: 24.4±6.3 pA; holding potential=−70 mV) and GABAergic synaptic events (frequency: 0.74±0.16 Hz; amplitude: 55.9±7.7 pA; holding potential=0 mV) (FIG. 12c, right traces) were recorded, consistent with the recovery of vGluT1 and GAD67 immunopuncta shown in FIG. 12b.

Significant tissue loss after severe stab injury during a variety of immunostaining analysis, but a remarkable tissue repair in the NeuroD1-treatment group (FIG. 11b) was observed. To quantitatively assess the level of tissue loss, serial cortical sections around the injury core in both mCherry control group and NeuroD1 treatment group were stained with Niss1. Niss1 staining revealed a large tissue loss across the serial brain sections in the mCherry control group after stab injury (FIG. 12d, top row). In contrast, the NeuroD1-treatment group exhibited much less tissue loss across all brain sections (FIG. 12d, bottom row). Quantitatively, the tissue loss around the injury areas in the NeuroD1 group was significantly reduced by 60% compared to the control group (FIG. 12d, right line graph, Two-way ANOVA, **P<0.01, n=5 pairs). Together, these results suggest that converting reactive astrocytes into neurons significantly reduced tissue loss and successfully repaired the damaged brain.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for repairing glial scar tissue in a cerebral cortex of a living mammal's brain, wherein said method comprises administering a nucleic acid sequence encoding a neuronal differentiation 1 (NeuroD1) polypeptide to astrocytes within said cerebral cortex, wherein said NeuroD1 polypeptide is expressed by said astrocytes, wherein said astrocytes form functional neurons within said cerebral cortex, and wherein said glial scar tissue is reversed back to neural tissue.

2. The method of claim 1, wherein said cerebral cortex, after administration of said nucleic acid sequence encoding a NeuroD1 polypeptide, has decreased expression of a factor selected from the group consisting of glial fibrillary acidic protein (Gfap), lipocalin-2 (Lcn2), chondroitin sulfate proteoglycan (CSPG), and combinations thereof.

3. The method of claim 1, wherein said cerebral cortex, after administration of said nucleic acid sequence encoding a NeuroD1 polypeptide, has increased expression of a factor selected from the group consisting of annexin A2 (Anax2), thrombospondin 1 (Thbs1), glypican 6 (Gpc6), brain-derived neurotrophic factor (Bdnf), and combinations thereof.

4-6. (canceled)

7. A method for reducing neuroinflammation in a cerebral cortex of a living mammal's brain, wherein said method comprises administering a nucleic acid sequence encoding a neuronal differentiation 1 (NeuroD1) polypeptide to astrocytes within said cerebral cortex, wherein said NeuroD1 polypeptide is expressed by said astrocytes, wherein said astrocytes form functional neurons within said cerebral cortex, wherein said neuroinflammation is reduced, and wherein said cerebral cortex has decreased expression of a factor selected from the group consisting of tumor necrosis factor alpha (TNFa), interleukin 1 beta (IL-1b), and cluster of designation 68 (CD68), and combinations thereof, after said administering.

8-10. (canceled)

11. A method for transforming an A1 astrocyte in a cerebral cortex of a living mammal's brain, wherein said method comprises administering a nucleic acid sequence encoding a neuronal differentiation 1 (NeuroD1) polypeptide to astrocytes within said cerebral cortex, wherein said NeuroD1 polypeptide is expressed by said astrocytes, wherein said astrocytes form functional neurons within said cerebral cortex, and wherein said A1 astrocyte is transformed into a less harmful astrocyte.

12. The method of claim 11, wherein said cerebral cortex, after said administering of said nucleic acid sequence encoding a NeuroD1 polypeptide, has decreased expression of a factor selected from the group consisting of guanylate Binding Protein 2 (Gbp2) serpin family G member 1 (Serping1), and combinations thereof.

13. A method for reducing the amount of toxic M1 microglia in a cerebral cortex of a living mammal's brain, wherein said method comprises administering a nucleic acid sequence encoding a neuronal differentiation 1 (NeuroD1) polypeptide to astrocytes within said cerebral cortex, wherein said NeuroD1 polypeptide is expressed by said astrocytes, wherein said astrocytes form functional neurons within said cerebral cortex, and wherein the amount of toxic M1 microglia is reduced.

14. The method of claim 13, wherein said toxic M1 microglia, after said administering of said nucleic acid sequence encoding a NeuroD1 polypeptide, have a morphology of resting microglia.

15. The method of claim 1, wherein said mammal is a human.

16. The method of claim 1, wherein said astrocytes are reactive astrocytes.

17-18. (canceled)

19. The method of claim 13, wherein said nucleic acid sequence encoding said NeuroD1 polypeptide is administered to said astrocytes in the form of a viral vector.

20. The method of claim 19, wherein said viral vector is an adeno-associated viral vector.

21-26. (canceled)

27. The method of claim 1, wherein said nucleic acid sequence encoding said NeuroD1 polypeptide is operably linked to a promoter sequence, wherein said promoter sequence is a constitutive promoter sequence.

28. (canceled)

29. The method of claim 1, wherein said administration comprises a direct injection into said cerebral cortex of said living mammal's brain.

30. The method of claim 1, wherein said administration comprises an intracranial, intrathecal, intraperitoneal, intravenous, intranasal, intramuscular, or oral administration.

31. A composition for forming functional neurons in a cerebral cortex of a living mammal's brain, wherein said composition comprises a) a nucleic acid vector comprising an inverted nucleic acid sequence encoding a neuronal differentiation 1 (NeuroD1) polypeptide flanked by recombinase target sites, and b) a nucleic acid vector comprising a nucleic acid sequence encoding a recombinase.

32. The composition of claim 31, wherein said nucleic acid vector comprising a nucleic acid sequence encoding a NeuroD1 polypeptide is a viral vector.

33. The composition of claim 32, wherein said viral vector is an adeno-associated viral vector.

34. (canceled)

35. The composition of claim 31, wherein said inverted nucleic acid sequence encoding said NeuroD1 polypeptide is operably linked to a promoter sequence, wherein said promoter sequence is a constitutive promoter sequence.

36-39. (canceled)

40. The composition of claim 31, wherein said nucleic acid sequence encoding said recombinase is operably linked to a promoter sequence, wherein said promoter sequence is an astrocyte-specific promoter sequence.

41. (canceled)

42. The composition of claim 31, wherein said recombinase is a Cre recombinase, and wherein said recombinase target sites are LoxP sites.

43-51. (canceled)

52. The method of claim 13, wherein said astrocytes are reactive astrocytes.
